(19) United States
(12) Patent Application Publication
(10) Pub. No.: US 2026/0306720 A1
(43) Pub. Date: Oct. 8, 2026

(54) SLIPPERY ORGANOGELS AND METHODS FOR MAKING AND USING THE SAME

(71) Applicant: University of Georgia Research Foundation, Inc., Athens, GA (US)

(72) Inventors: Hitesh Handa, Athens, GA (US); Elizabeth Brisbois, Athens, GA (US); Isabel Martinez, Athens, GA (US); Arpita Shome, Athens, GA (US)

(21) Appl. No.: 19/639,446

(22) Filed: Apr. 6, 2026

Related U.S. Application Data
(60) Provisional application No. 63/785,049, filed on Apr. 8, 2025.

(51) Int. Cl. A61L 31/14; A61L 31/10; A61L 31/16

(52) U.S. Cl. CPC A61L 31/145; A61L 31/10; A61L 31/16; A61L 2420/02

(57) ABSTRACT

In accordance with the purpose(s) of the present disclosure, as embodies and broadly described herein, the disclosure, in one aspect, relates to slippery organogels and a method of making nitric oxide releasing compositions. The slippery organogels include (a) compound comprising the reaction product between a polysiloxane comprising one or more isocyanate reactive groups and a diisocyanate and (b) a lubricant. The mechanical properties of the organogels can be modified depending upon their application. The ability of the slippery organogels to prevent adhesion of platelets and proteins as well as prevent the formation of biofilms make them suitable for the production of medical devices to prevent microbial infections.

### CROSS-REFERENCE TO RELATED APPLICATIONS

This application claims the benefit of and priority to co-pending U.S. Provisional Patent Application No. 63/785,049, filed on Apr. 8, 2025, the contents of which are incorporated by reference herein in their entireties.

### STATEMENT REGARDING FEDERALLY SPONSORED RESEARCH OR DEVELOPMENT

This invention was made with government support under grant number R01 HL151473 awarded by the National Institutes of Health. The Government has certain rights in the invention.

### BACKGROUND

Organogels are three-dimensional (3D) networks consisting of organic liquids that are marked with improved mechanical, thermal and environmental stability over hydrogels with widespread applications in droplet manipulation, drug delivery, anti-fouling, flexible sensors etc. The 3D network is mainly made up of stimuli-responsive polymers^{4, 5}, supramolecular polymers and polymer-nanoparticles composites. The surface properties of organogels are determined by the solid matrix as well as the infused organic liquid³. In that context, infusion with lubricants such as silicone oil and paraffin wax impart the Nepenthes Pitcher plant inspired slippery properties to the organogel, commonly termed as slippery organogels. However, solid lubricant cannot be used in Nepenthes Pitcher plant inspired slippery surfaces since paraffin wax solidifies at room temp and we need the infused lubricant to remain in the liquid form.

However, the widely reported slippery, anti-biofouling organogels suffer from general limitations such as a) complex fabrication, b) lack of toxicity analysis, c) lack of prolonged chemical durability, e) inflexibility to use green solvents, and f) complex fabrication, that restricts their biomedical utility. Moreover, to date none of the reported slippery organogels have been quantified in detail for protein and platelet repellent ability or hemolysis. Furthermore, the anti-biofouling characteristics of such slippery organogels are primarily dependent on the infused lubricant layer (passive barrier) which is prone to depletion, volatilization and migration overtime.

### SUMMARY

In accordance with the purpose(s) of the present disclosure, as embodies and broadly described herein, the disclosure, in one aspect, relates to slippery organogels and a method of making nitric oxide releasing compositions. The slippery organogels include (a) compound comprising the reaction product between a polysiloxane comprising two or more isocyanate reactive groups and a diisocyanate and (b) a lubricant. The mechanical properties of the organogels can be modified depending upon their application. The ability of the slippery organogels to prevent adhesion of platelets and proteins as well as prevent the formation of biofilms make them suitable for the production of medical devices to prevent microbial infections.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A-1K. (A-C) Schematic depicting the polycondensation between the amines of bis(3-aminopropyl) terminated poly dimethylsiloxane (bis-APT-PDMS) with the isocyanate groups of isophorone diisocyanate (IPDI) to obtain a siloxane based slippery organogel after infusion with silicone oil as the lubricant. (D-E) Digital image (D) and contact angle image (E) depicting the organogel coating on a polyvinyl chloride polymer (PVC) without any slippery characteristics. (F-I) Digital image (F-H) and contact angle image (G-I) depicting the sliding of water droplet from the PVC surface after infusion of the organogel coating with silicone oil. (J) Graph accounting for the variation in water contact angles and water sliding angles with respect to the PDMS and IPDI ratio. (K) The reaction between the bis-APT-PDMS and IPDI was monitored through Fourier Transform Infrared (FTIR) spectrometer via the disappearance of the NCO stretching peak and appearance of the C═O stretching vibration peak indicative of ureido bond formation.

FIGS. 2A-21. (A) Schematic depicting the incorporation of the anti-thrombotic and antibacterial gasotransmitter, S-nitroso-N-acetyl-D-penicillamine (SNAP) into the PVC substrate abbreviated as PVC-SNAP. B-C) Digital image (B) and contact angle image (C) of a water droplet pinned on PVC-SNAP. D-G) Digital images (D,F) and contact angle images (E,G) of a water droplet sliding from the PVC-SNAP coated with the organogel (abbreviated as PVC-SNAP-OGL) followed by infusion with silicone oil to obtain a lubricant infused slippery, nitric oxide releasing polymeric material abbreviated as PVC-SNAP-LIS. (H) Plot depicting the nitric oxide (NO) release over 48 h under physiological conditions (pH=7.4, 37° C.) from PVC-SNAP, PVC-SNAP-OGL and PVC-SNAP-LIS. (I) Graph accounting for the leaching of the SNAP molecule from PVC-SNAP, PVC-SNAP-OGL and PVC-SNAP-LIS over 48 h under physiological conditions.

FIGS. 3A-3H. (A-B) Schematic illustrating the (A) ureido group formation and dynamic hydrogen bonds through the polycondensation reaction of the amine groups of bis-APT-PDMS and isocyanate groups of IPDI. (B) The dynamic bonds are primarily responsible for the self-healing characteristics of the organogel coating. (C-D) Optical microscopy images of PVC-LIS (C) initially after an incision is made on the surface and (D) the healed surface after 12 h. (E-F) Optical microscopy images of PVC-SNAP-LIS (E) initially after an incision is made on the surface and (F) the healed surface after 12 h. (G) Graph representing the water sliding behavior over 24 h following incision on PVC-LIS and PVC-SNAP-LIS. H) Graph depicting the water sliding behavior over 24 h following incision and healing at 37° C. incubation for PVC-LIS and PVC-SNAP-LIS.

FIGS. 4A-4D. Graph presenting the water sliding angles on PVC-LIS and PVC-SNAP-LIS after UV irradiation (A), PBS immersion (B), repetitive tape peeling cycles (C) and prolonged room temperature exposure (D).

FIGS. 5A-5I. (A-B) Graph illustrating the S. aureus (A) and E. coli (B) and biomass accumulation on the different sample types following a 48 h drip flow bioreactor experiment. C) Graph depicting the relative cell viability of the different sample types against human umbilical endothelial cells (HUVECs). (D-G) Fluorescence microscopy images depicting the adherence of fluorescein-labelled fibrinogen on the different sample types. (H) Graph accounting for the platelets adhered onto the surface of the different polymeric control samples and the combinatorial material (PVC-SNAP-LIS). (1) The hemolytic index is depicted for the different polymeric materials. *p≤0.01, **p≤0.01, ***p≤0.001, ****p≤0.0001 and ns is non-significant. All data are represented as mean±SD.

FIGS. 6A-6H. (A-D) Digital images of ECC loops following 4 h of blood flow through A-B) PVC and C-D) PVC-SNAP-LIS loops. (E) Graph depicting the platelet aggregation following collagen activation throughout the 4 h ECC study. (F-H) Graphs presenting the (F) platelets remaining in the blood compared to baseline concentrations (G) thrombus mass formed in the thrombogenicity chamber and H) thrombus area calculated on thrombogenicity chamber following 4 h of blood flow through the loops. *p≤0.01, **p≤0.01, ***p≤0.001, ****p≤0.0001 and ns is non-significant. All data are represented as mean±SD.

FIGS. 7A-7N. (A-B) Digital image (A) and sliding angle image (B) depicting the restricted sliding of a water droplet on an Elast-eon polymer. (C-F) Digital images (C, E) and water contact angle images (D, F) depicting the sliding of water droplets on the organogel coated lubricant infused coating on Elast-eon. (G-H) Digital image (G) and sliding angle image (H) depicting the restricted sliding of a water droplet on a room temperature vulcanizing (RTV) polymer. (I-L) Digital images (1, K) and water contact angle images (J, L) depicting the sliding of water droplets on the organogel coated lubricant infused coating on RTV. (M) Water sliding behavior after room temperature storage for 60 days (n=4). (N) Tape peel test conducted on Elast-eon and RTV for 50 cycles (n=4).

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions and Abbreviations

In describing and claiming the disclosed subject matter, the following terminology will be used in accordance with the definitions set forth below.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent” includes, but are not limited to, mixtures or combinations of two or more such solvents, and the like.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed and “about 5 to about 15” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

The term “alkyl group” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

In some embodiments, a straight chain or branched chain alkyl group has 30 or fewer carbon atoms in its backbone (e.g., C₁-C₃₀ for straight chains, C₃-C₃₀ for branched chains), 20 or fewer, 12 or fewer, or 7 or fewer.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkylene” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The alkylene group is represented by the formula —(CH₂)ₙ—, where n is an integer from 1 to 10. The groups, —CH₂— (methylene), —CH₂CH₂—, —CH₂C(CH₃)₂CH₂—, and —CH₂CH₂CH₂— are non-limiting examples of alkylene groups.

The term “alkylene cycloalkyl group” as used herein, refers a cycloalkyl group as defined herein includes one or more alkylene groups as defined herein covalently bonded to the cycloalkyl group. The point of attachment is to the alkylene group and not the cycloalkyl group. For example, in ROH where R is an alkylene cycloalkyl group, the alkylene group is bonded to oxygen.

As used herein, the term “biocompatible,” with respect to a substance or fluid described herein, indicates that the substance or fluid does not adversely affect the short-term viability or long-term proliferation of a target biological particle within a particular time range.

The terms “antimicrobial” and “antimicrobial characteristic” refer to the ability to kill and/or inhibit the growth of microorganisms. A substance having an antimicrobial characteristic may be harmful to microorganisms or microbes (e.g., bacteria, fungi, virus, protozoans, algae, and the like). A substance having an antimicrobial characteristic can kill the microorganism and/or prevent or substantially prevent the growth or reproduction of the microorganism.

The terms “bacteria” or “bacterium” include, but are not limited to, gram positive and gram negative bacteria. Bacteria can include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anabaena affinis and other cyanobacteria (including the Anabaena, Anabaenopsis, Aphanizomenon, Camesiphon, Cylindrospermopsis, Gloeobacter Hapalosiphon, Lyngbya, Microcystis, Nodularia, Nostoc, Phormidium, Planktothrix, Pseudoanabaena, Schizothrix, Spirulina, Trichodesmium, and Umezakia genera) Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Phytoplasma, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Spiroplasma, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholera, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof. The gram-positive bacteria may include, but is not limited to, gram positive Cocci (e.g., Streptococcus, Staphylococcus, and Enterococcus). The gram-negative bacteria may include, but is not limited to, gram negative rods (e.g., Bacteroidaceae, Enterobacteriaceae, Vibrionaceae, Pasteurellae and Pseudomonadaceae).

The terms “fungus” or “fungi” include, but are not limited to yeasts such as, for example, Candida albicans or other Candida spp. including C. glabrata, C. rugosa, C. parapsilosis, C. tropicalis, or C. dubliniensis Fungi can also include dermatophytes such as, for example, Trichophyton spp. and Microsporum spp. (e.g., T. rubrum, T. interdigitale, T. tonsurans, T. violaceum, T. concentricum, T. schoenleinii, T. soudanense, T. mentagrophytes, T. equinum, T. erinacei, T. verrucosum, M. audouinii, M. ferrugineum, M. canis, M. gypseum, M. nanum, and/or M. cookie).

The term “antimicrobial effective amount” as used herein refers to that amount of the compound being administered/released that will kill microorganisms or inhibit growth and/or reproduction thereof to some extent (e.g. from about 5% to about 100%). In reference to the compositions or articles of the disclosure, an antimicrobial effective amount refers to that amount which has the effect of diminishment of the presence of existing microorganisms, stabilization (e.g., not increasing) of the number of microorganisms present, preventing the presence of additional microorganisms, delaying or slowing of the reproduction of microorganisms, and combinations thereof. Similarly, the term “antibacterial effective amount” refers to that amount of a compound being administered/released that will kill bacterial organisms or inhibit growth and/or reproduction thereof to some extent (e.g., from about 5% to about 100%). In reference to the compositions or articles of the disclosure, an antibacterial effective amount refers to that amount which has the effect of diminishment of the presence of existing bacteria, stabilization (e.g., not increasing) of the number of bacteria present, preventing the presence of additional bacteria, delaying or slowing of the reproduction of bacteria, and combinations thereof.

As used herein, the term “subject” includes humans, mammals (e.g., cats, dogs, horses, etc.), birds, and the like. Typical subjects to which embodiments of the present disclosure may be administered will be mammals, particularly primates, especially humans. For veterinary applications, a wide variety of subjects will be suitable, e.g., livestock such as cattle, sheep, goats, cows, swine, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “reduce” or “reducing” refers to deceasing the degree or amount of an event. For example, reducing biofilm formation can mean lower the amount of biofilm formation when using the compositions described herein when compared to the amount of biofilm formation when the compositions described herein are not used.

### Slippery Organogels and Preparation Thereof

In accordance with the purpose(s) of the present disclosure, as embodies and broadly described herein, the disclosure, in one aspect, relates to nitric oxide releasing slippery organogels and a method of making nitric oxide releasing compositions. The slippery organogels include (a) compound comprising the reaction product between a polysiloxane comprising two or more isocyanate reactive groups and a diisocyanate and (b) a lubricant.

The reaction between the polysiloxane comprising two or more isocyanate reactive groups and diisocyanate results in the formation of an organogel without the need for any catalyst.

In one aspect, the slippery organogel is produced by the method comprising mixing the reaction product between a polysiloxane comprising two or more isocyanate reactive groups and a diisocyanate and lubricant. In another aspect, the reaction product between a polysiloxane comprising two or more isocyanate reactive groups and a diisocyanate and the lubricant can be sequentially applied to an article to produce slippery organogel. The Examples provide non-limiting procedures for making the organogels described herein.

The polysiloxane useful herein includes two or more isocyanate reactive groups. In one aspect, the isocyanate reactive group includes a hydroxyl group, a thiol group, or an amino group.

In one aspect, the polysiloxane is a linear or branched polysiloxane, wherein each end of the linear or branched polysiloxane has an isocyanate reactive group covalently bonded to each end. In one aspect, the polysiloxane with two or more isocyanate reactive groups includes a polydimethylsiloxane, a polydiethylsiloxane, a polydipropylsiloxane, or a polydiphenylsiloxane.

In one aspect, the polysiloxane with two or more isocyanate reactive groups has the structure I

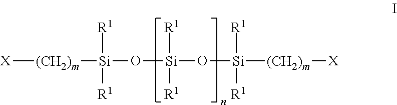

wherein each R¹ is independently selected from alkyl, aryl, alkenyl, hydrogen, alkoxy;
m is an integer from 1 to 10;
n is from about 2,500 to about 27,000; and
X is an isocyanate reactive group.

In one aspect, each R¹ in structure I is a methyl group. In another aspect, each m in structure I is 3. In another aspect, n in structure I is 2,500, 5,000, 7,500, 10,000, 12,500, 15,000, 17,500, 20,000, 22,500, 25,00, or 27,000, where any value can be a lower and upper endpoint of a range (e.g., 5,000 to 17,500).

The diisocyanate that reacts with the polysiloxane with two or more isocyanate reactive groups to produce the organogels described herein is a molecule that possesses two isocyanate groups. In one aspect, the diisocyanate has the formula O═C═N—R¹—N═C═O, wherein R¹ is an alkylene group, a cycloalkyl group, an alkylene cycloalkyl group, or an aryl group.

In one aspect, the diisocyanate comprises toluene-2,4-diisocyanate (TDI), diphenylmethane diisocyanate, methylene diphenyl diisocyanate (MDI), hexarnethylene diisocyanate (HMDI), 2,2,4-trirnethylhexane-1,6-diisocyanate (TMDI), isophorone diisocyanate (IPDI), or 4,4′-diisocyanatodicyclohexylnethane (H12MDI).

In one aspect, the molar ratio of the polysiloxane comprising two or more isocyanate reactive groups and the isocyanate is from 3:1 to 0.5:1. In another aspect, the molar ratio of the polysiloxane comprising two or more isocyanate reactive groups and the diisocyanate is 3:1, 2.5:1, 2:1, 1.5:1, 1:1, or 0.5:1, where any value can be a lower and upper endpoint of a range (e.g., 3:1 to 1.5:1). In another aspect, the molar ratio of the polysiloxane comprising two or more isocyanate reactive groups and the diisocyanate is 1.25:1 to 0.75:1, or about 1:1.

In one aspect, the lubricant is an oil such as, for example, silicone oil or any other biocompatible oil. The amount of lubricant used to produce the slippery organogel can vary depending upon the article to be coated with the organogel. In one aspect, the amount of lubricant is sufficient to coat the entire article or specific portion or section so the article.

In one aspect, depending upon the application of the slippery organogel, the organogel can include one or more additional bioactive agents including, but not limited to, an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof. The bioactive agent can be incorporated into the slippery organogel during any phase of production.

In one aspect, the bioactive agent is a nitric oxide releasing compound. The nitric oxide releasing compound is a compound that possesses one or more nitric oxide groups, wherein nitric oxide can be released from the organogel.

In one aspect, the nitric oxide releasing compound is an S-nitrosothiol (RSNO) compound. In a further aspect, the nitric oxide compound is S-nitroso-N-acetylpenicillamine, S-nitroso-glutathione, S-nitroso-N-acetylcysteine, S-nitrosocysteine, S-nitrosopenicillamine, S-nitroso-B,D-glucose, S-nitrosocaptopril, S-nitrosocysteamine, S-nitroso-3-mercapto-propanoic acid, S-nitroso-N-acetyl-1-cysteine ethyl ester (SNACET), S-nitroso-N-acetyl-L-methionine, S-nitrosomercaptoethanol, or any combination thereof. In another aspect, the nitric oxide releasing compound can be S-nitrosothiol conjugated polymers, S-nitrosothiol modified-dendrimers, S-nitrosothiol modified polysaccharides, S-nitrosothiol modified nano/microparticles, or S-nitrosothiol modified-proteins. The nitric oxide releasing compound can also include other NO-donors such as, for example, nitrates and N-diazeniumdiolates (NONOates).

In other aspects, the nitric oxide releasing compound includes a modified antibiotic compound including a nitric oxide release agent covalently attached to an antibiotic molecule. Having a single molecule with the combined functionalities of both of a stable NO donor and an antibiotic can be a very efficient approach for combating and preventing biofilm related infections. The modified antibiotic compound can be a synthetic RSNO covalently attached to an antibiotic molecule to create a novel dual functional antimicrobial agent, also referred to as a modified antibiotic compound. In other aspects, the nitric oxide releasing agent is S-nitroso-N-acetylpenicillamine (SNAP), S-nitroso-glutathione, S-nitroso-N-acetylcysteine, S-nitrosocysteine, S-nitrosopenicillamine, S-nitroso-B,D-glucose, S-nitrosocaptopril, S-nitrosocysteamine, S-nitroso-3-mercapto-propanoic acid, or any combination thereof. In other aspects, the antibiotic molecule can be ampicillin, vancomycin, gentamicin, cephalexin, or any combination thereof. In further aspects, the modified antibiotic compound includes SNAP covalently bonded to ampicillin, referred to herein as SNAPicillin. SNAP can be represented by the following structure:

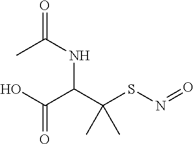

SNAPicillin can be represented by the following structure:

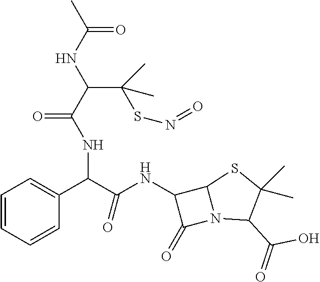 [SMILES: CC(=O)NC(C(=O)NC(C(=O)NC1C(=O)N2C1SC(C)(C)C2C(=O)O)c1ccccc1)C(C)(C)SN=O]

In one aspect, the modified antibiotic compound can be formed by covalently attaching a nitric oxide release agent to an antibiotic molecule. The attachment can be formed by mixing the nitric oxide release agent and the antibiotic molecule in a solvent and then nitrosating the mixture. The nitrosation can occur through the excess addition of t-butyl nitrite or an acidified sodium nitrite solution to the mixture. The excess addition can be about a 3 times molar excess of t-butyl nitrate with respect to the quantity of the antibiotic molecule, such as ampicillin. Methods for producing the modified antibiotic compound useful as nitric oxide releasing compounds are described in U.S. Pat. No. 11,220,516, which is incorporated by reference in its entirety.

The amount of the nitric oxide releasing compound incorporated into the organogels described herein can vary. For example, when preparing the organogel, the reaction product between the reaction product between a polysiloxane comprising two or more isocyanate reactive groups and a diisocyanate can be contacted with a composition comprising the nitric oxide releasing compound and an organic solvent. In one aspect, the reaction product between a polysiloxane comprising two or more isocyanate reactive groups and a diisocyanate can be contacted with a nitric oxide releasing compound in an organic solvent, where the concentration of the nitric oxide releasing compound is from 5 mg/mL to 50 mg/mL. In one aspect, the organic solvent is an alcohol, (e.g., methanol, ethanol), acetone, or tetrahydrofuran.

The organogels described herein possess good slippery properties, which is advantageous in numerous applications such as, for example, coating medical devices. In one aspect, the organogel has a water static contact angle of greater than 60 degrees. In another aspect, the organogel has a water static contact angle of 60 degrees, 65 degrees, 70 degrees, 75 degrees, 80 degrees, 85 degrees, or 90 degrees, where any value can be a lower and upper endpoint of a range (e.g., 60 degrees to 80 degrees). In one aspect, the organogel has a water sliding angle of less than 20 degrees. In another aspect, the organogel has a water sliding angle of 6 degrees, 8 degrees, 10 degrees, 12 degrees, 14 degrees, 16 degrees, 18 degrees, or less than 20 degrees, where any value can be a lower and upper endpoint of a range (e.g., 12 degrees to 18 degrees). Methods for determining the water sliding contact angle and the water static contact angle of the organogels described herein are provided in the Examples.

In addition to good slippery properties, the organogel possess several other desirable properties making them suitable for biomedical applications. For biomedical applications, self-healing polymeric networks that do not require the use of external stimuli are desirable as the use of excessive heat or UV irradiation can cause harm to the surrounding tissue and limits the loading of stimuli-sensitive drug molecules. As demonstrated herein the organogels are capable of self-healing at room temperature.

The organogels described herein are durable materials that can be stored for extended periods of time. As demonstrated herein, the organogels can withstand handling and storage conditions, as well as sterilization methods. The organogels maintain good slippery properties when stored for extended periods of time (e.g., up to 60 days). Similarly, organogels maintain good slippery properties when exposed to UV irradiation at 365 nm for 30 min which is a common sterilization method prior to using medical devices for clinical applications.

The organogels described herein can be used in applications where the organogel is used in clinical blood-contacting applications. A hemolytic index of 0-2% is considered non-hemolytic, 2-5% considered slightly hemolytic, and >5% hemolytic. The organogels described herein have a hemolytic index of about 1±0.2%, thus classified as non-hemolytic. The organogels described herein are suitable for blood-contacting device applications.

### Applications of Organogels

The organogels possess unique properties that make them suitable in a variety of different applications. The organogels described herein are biocompatible, which make them useful in biomedical applications. The organogels can impart slippery coatings on medical polymers, which can aid in inhibiting the adhesion of biomolecules or microbes on the surface of a medical devices. Thus, the ease of application and substrate-independent characteristics of the organogels described herein coating qualifies it for a wide range of biomedical, energy, and environmental applications ranging from anti-biofouling, oil-water separation, biosensors, and wound dressing.

In one aspect, an article or surface of an article can be coated with an organogel described herein where it is desirable to reduce or prevent biofouling (e.g. growth of bacteria, adhesion of platelets, adhesion of fibrinogen). Biofilm and thrombus formation on surfaces results in significant morbidity and mortality worldwide, which highlights the importance of the development of efficacious fouling-prevention approaches.

In one aspect, the organogels described herein are useful in applications where it is desirable to reduce or prevent biofouling. Implantable medical devices are a leading cause of infection such as nosocomial infections. Implantable devices coated with or constructed of the compositions described herein can reduce or prevent biofouling in a subject when the device is introduced into the subject. In one aspect, the organogels described herein can reduce or prevent bacterial growth on a surface of an implantable device. In another aspect, the compositions described herein can reduce or prevent biofilm formation on a surface of an implantable device. In another aspect, the compositions described herein can reduce or prevent fibrinogen formation on a surface of an implantable device.

In one, the implantable device is a urinary catheter, artificial heart valve, a vascular catheter, a graft, or a stent. In other aspects, the device is intended to contact human blood or tissue. In one aspect, the device is a hemodialysis device or a component thereof. The coating compositions described herein are biocompatible (e.g., with fibroblast cells), which makes them useful in implantable medical devices. In another aspect, article is a wound dressing including, but not limited to a bandage, gauze, pad.

In one aspect, the device to be coated is composed of a medical grade polymer. Examples of such polymers include, but are not limited to a polycarbonate, a polypropylene copolymer or homopolymer, a polyethylene copolymer or homopolymer, polyvinylchloride, polystyrene, polyethylene terephthalate glycol, polymethyl methacrylate, a polyetheretherketone, polymethylpentene, a polyurethane-siloxane (e.g., Elasteon), a polyurethane, or a polysiloxane.

The organogels described herein can be incorporated into devices in a number of different ways. In one aspect, the devices can be coated with the organogels described herein. The coating thickness can vary as well depending upon the device and application selected. In one aspect, the coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In one aspect, the slippery organogels can include a nitric oxide releasing compound as described above. In other aspects, the article can include a nitric oxide releasing compound, where the article is subsequently coated with the organogel as described herein. In one aspect, one or more components of an article can be fabricated with the nitric oxide releasing compound. In one aspect, a polymer mixture composed of the nitric oxide releasing compound can be poured into a mold. When a solvent is present in the composition, the solvent can be removed to produce a molded article composed of the nitric oxide releasing compound dispersed throughout the article.

In another aspect, the organogel can be applied sequentially to a surface of an article. For example, the compound comprising the reaction product between a polysiloxane comprising two or more isocyanate reactive groups and a diisocyanate can be first applied to the article followed by the application of the lubricant. In another aspect, the nitric oxide releasing compound can be applied to a surface of an article followed the application of the compound comprising the reaction product between a polysiloxane comprising two or more isocyanate reactive groups and a diisocyanate and then the lubricant. The organogel and nitric oxide releasing compound can be applied to the article using techniques known in the art such as, for example, dipping or spraying.

In one aspect, the organogels described herein are useful in applications where it is desirable to reduce or prevent biofouling on polymeric medical grade materials. In other aspects, the organogels described herein are useful in applications where it is desirable to reduce or prevent biofouling on metals (e.g. steel, aluminum). In other aspects, the organogels described herein are useful in applications where it is desirable to reduce or prevent biofouling on hospital touch surfaces (e.g. bed rails, bed frames, and handles). In other aspects, the organogels described herein are useful in applications where it is desirable to reduce or prevent biofouling on an automobile surfaces boat hull, or aircraft.

In one aspect, the organogels described herein can prevent the growth of bacteria on an article, in which the method includes applying the organogel as above to at least one surface of the article. Bacterial infections of blood-contacting medical devices accounts for majority of the hospital acquired infections and fatalities in the United States. As antibiotic resistance and failure of antibiotics to penetrate biofilms is a growing concern globally, a broad-spectrum antimicrobial agent having multiple mechanisms of action is desirable that can significantly reduce the development of resistance among microbes. In one aspect, the organogels described herein can prevent about at least 70%, at least 75%, at least 80%, at least 85%, at least 90%, at least 95%, at least 99%, or 100% of the growth of bacteria when compared to an uncoated article.

In one aspect, the organogels described herein can prevent the adhesion of fibrinogen on an article, in which the method includes applying the organogel as above to at least one surface of the article. The adsorption of proteins onto the surface of medical devices is the initial step in surface-induced coagulation that is influenced by surface morphology and chemistry. Minimizing the adsorption of fibrinogen reduces the risk of thrombotic complications which can prolong the use of the medical device and ensure patient safety. The organogels described herein can prevent about at least 70%, at least 75%, at least 80%, at least 85%, at least 90%, at least 95%, at least 99%, or 100% of the adhesion of fibrinogen when compared to an uncoated article.

In one aspect, the organogels described herein can prevent the adhesion of platelets on an article, in which the method includes applying the organogel as above to at least one surface of the article. Platelet adhesion and subsequent activation on blood-contacting biomaterial surfaces initiates coagulation and eventually leads to thrombus formation. Overcoming this phenomenon is important in developing a hemocompatible biomaterial surface. The organogels described herein can prevent about at least 70%, at least 75%, at least 80%, at least 85%, at least 90%, at least 95%, at least 99%, or 100% of the adhesion of platelets when compared to an uncoated article.

### Aspects

Aspect 1. A slippery organogel comprising (a) compound comprising the reaction product between a polysiloxane comprising two or more isocyanate reactive groups and a diisocyanate and (b) a lubricant.

Aspect 2. The slippery organogel of Aspect 1, wherein the polysiloxane comprises a linear or branched polysiloxane, wherein each end of the linear or branched polysiloxane has an isocyanate reactive group covalently bonded to each end.

Aspect 3. The slippery organogel of Aspect 1 or 2, wherein the polysiloxane comprises a polydimethylsiloxane, a polydiethylsiloxane, a polydipropylsiloxane, or a polydiphenylsiloxane.

Aspect 4. The slippery organogel of Aspect 1, wherein the polysiloxane has the structure I

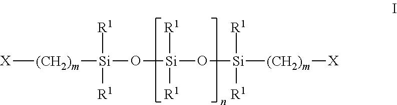

wherein each R¹ is independently selected from alkyl, aryl, alkenyl, hydrogen, alkoxy;
m is an integer from 1 to 10;
n is from about 2,500 to about 27,000; and
X is an isocyanate reactive group.

Aspect 5. The slippery organogel of Aspect 4, wherein each R¹ is a methyl group.

Aspect 6. The slippery organogel of Aspect 4 or 5, wherein each m is 3.

Aspect 7. The slippery organogel of any one of Aspects 4-6, wherein each X is NH₂.

Aspect 8. The slippery organogel of any one of Aspects 1-7, wherein the isocyanate reactive group comprises a hydroxyl group, a thiol group, or an amino group.

Aspect 9. The slippery organogel of any one of Aspects 1-8, wherein the diisocyanate has the formula O═C═N—R¹—N═C═O, wherein R¹ is an alkylene group, a cycloalkyl group, an alkylene cycloalkyl group, or an aryl group.

Aspect 10. The slippery organogel of Aspect 9, wherein R¹ is an alkylene cycloalkyl group,

Aspect 11. The slippery organogel of any one of Aspects 1-10, wherein the diisocyanate comprises toluene-2,4-diisocyanate (TDI), diphenylmethane diisocyanate, methylene diphenyl diisocyanate (MDI), hexamethylene diisocyanate (HMDI), 2,2,4-trimethylhexane-1,6-diisocyanate (TMDI), isophorone diisocyanate (IPDI), or 4,4′-diisocyanatodicyclohexylmethane (H12MDI).

Aspect 12. The slippery organogel of any one of Aspects 1-10, wherein the diisocyanate comprises isophorone diisocyanate.

Aspect 13. The slippery organogel of any one of Aspects 1-12, wherein the molar ratio of polysiloxane comprising two or more isocyanate reactive groups to the diisocyanate is from 3:1 to 0.5:1.

Aspect 14. The slippery organogel of any one of Aspects 1-12, wherein the molar ratio of polysiloxane comprising two or more isocyanate reactive groups to the diisocyanate is from 1.25:1 to 0.75:1.

Aspect 15. The slippery organogel of any one of Aspects 1-12, wherein the molar ratio of polysiloxane comprising two or more isocyanate reactive groups to the diisocyanate is from about 1:1.

Aspect 16. The slippery organogel of any one of Aspects 1-15, wherein the lubricant comprises an oil.

Aspect 17. The slippery organogel of any one of Aspects 1-15, wherein the lubricant comprises a silicone oil.

Aspect 18. The slippery organogel of any one of Aspects 1-17, wherein the organogel further comprises a nitric oxide releasing compound.

Aspect 19. The slippery organogel of Aspect 18, wherein the nitric oxide releasing compound is a S-nitrosothiol conjugated polymer, a S-nitrosothiol modified-dendrimers; a S-nitrosothiol modified polysaccharide, a S-nitrosothiol modified nano/microparticle, a S-nitrosothiol modified-protein, a nitrate, a N-diazeniumdiolates (NONOate), or a S-nitrosothiol (RSNO).

Aspect 20. The slippery organogel of Aspect 18, wherein the nitric oxide releasing compound is a modified antibiotic compound comprising a nitric oxide release agent covalently attached to an antibiotic molecule

Aspect 21. The slippery organogel of Aspect 18, wherein the nitric oxide release agent is S-nitroso-N-acetylpenicillamine, S-nitroso-glutathione, S-nitroso-N-acetylcysteine, S-nitrosocysteine, S-nitrosopenicillamine, S-nitroso-B,D-glucose, S-nitrosocaptopril, S-nitrosocysteamine, and S-nitroso-3-mercapto-propanoic acid.

Aspect 22. The slippery organogel of Aspect 20 or 21, wherein the antibiotic molecule is ampicillin, vancomycin, gentamicin, or cephalexin.

Aspect 23. The slippery organogel of Aspect 22, wherein the modified antibiotic compound comprises S-nitroso-N-acetylpenicillamine covalently attached to ampicillin.

Aspect 24. The slippery organogel of Aspect 18, wherein the nitric oxide releasing compound is a S-nitrosothiol compound.

Aspect 25. The slippery organogel of Aspect 18, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, S-nitroso-N-acetyl-l-cysteine ethyl ester (SNACET), S-nitroso-L-homocysteine, S-nitroso-L-cysteine, S-nitroso-albumin, S-nitrosocaptopril, or any combination thereof.

Aspect 26. The slippery organogel of Aspect 18, wherein the nitric oxide releasing compound is S-nitroso-N-acetyl-penicillamine.

Aspect 27. The slippery organogel of any one of Aspects 1-26, wherein the slippery organogel further comprises a bioactive agent.

Aspect 28. The slippery organogel of Aspect 27, wherein the bioactive agent comprises an antibiotic, a pain reliever, an immune modulator, a growth factor, an enzyme inhibitor, a hormone, a messenger molecule, a cell signaling molecule, a receptor agonist, an oncolytic virus, a chemotherapy agent, an anti-angiogenic agent, a receptor antagonist, a nucleic acid, or any combination thereof.

Aspect 29. The slippery organogel of any one of Aspects 1-28, wherein the slippery organogel has a water sliding contact angle of less than 20 degrees.

Aspect 30. The slippery organogel of any one of Aspects 1-28, wherein the slippery organogel has a water static contact angle of greater than 80 degrees.

Aspect 31. An article comprising at least one surface coated with the slippery organogel of any one of Aspects 1-30.

Aspect 32. The article of Aspect 31, wherein the article comprises a polymeric grade material, a medical device, a surface or article in a hospital or medical facility, a wound dressing, or a surface in an automobile, boat, or aircraft.

Aspect 33. The article of Aspect 31, wherein the article comprises a medical device.

Aspect 34. The article of Aspect 31, wherein the article is an implantable device.

Aspect 35. The article of Aspect 31, wherein the article is a catheter, a cannula, tubing, a suture, an artificial heart valve, a graft, a stent, circuitry, or a joint prostheses.

Aspect 36. The article of Aspect 31, wherein the article comprises a dressing.

Aspect 37. The article of any one of Aspects 31-36, wherein the article comprises a polycarbonate, a polypropylene copolymer or homopolymer, a polyethylene copolymer or homopolymer, polyvinylchloride, polystyrene, polyethylene terephthalate glycol, polymethyl methacrylate, a polyetheretherketone, polymethylpentene, a polyurethane-siloxane, a polyurethane, or a polysiloxane.

Aspect 38. The article of any one of Aspects 31-37, wherein the article comprises a nitric oxide releasing compound at the surface of the article, dispersed throughout the article, or a combination thereof.

Aspect 39. A method for reducing or preventing the growth of microbes on an article, the method comprising applying the slippery organogel of any one of Aspects 1-30 to at least one surface of the article.

Aspect 40. A method for reducing or preventing the formation of a biofilm on an article, the method comprising applying the slippery organogel of any one of Aspects 1-30 to at least one surface of the article.

Aspect 41. A method for reducing or preventing the adhesion of platelets on an article, the method comprising applying the slippery organogel of any one of Aspects 1-30 to at least one surface of the article.

Aspect 42. A method for reducing or preventing the adhesion of a protein on an article, the method comprising applying the slippery organogel of any one of Aspects 1-30 to at least one surface of the article.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Materials and Methods

### Materials

N-Acetyl-D-penicillamine (NAP), polyvinyl chloride (high molecular weight), trioctyl trimellitate (TO™), bis(3-aminopropyl) terminated poly dimethylsiloxane (bis-APT-PDMS) (Mn: 2,500), Isophorone Diisocyanate (IPDI), tetrahydrofuran (THF), silicone oil (100 cSt), hydrochloric acid (HCl), hydrogen peroxide (H₂O₂), sulfuric acid (H₂SO₄), magnesium chloride (MgCl₂), magnesium sulphate (MgSO₄), sodium chloride (NaCl), calcium chloride (CaCl₂), nitric acid, sodium citrate dihydrate, triton-X, phosphate buffer saline (PBS), Yellow 3-(4,5 dimethylthiazol-2-yl)-2,5-diphenyl-2H-tetrazolium bromide (MTT), Roche LDH cytotoxicity assessment kit, Luria-Bertani (LB) broth and agar from Sigma-Aldrich (St. Louis MO, USA). Elast-eon 5-325 was obtained from Biomerics (Salt Lake City Utah, USA). DOW Dowsil 3140 room temperature vulcanized siloxane (RTV) was procured from Ellsworth Adhesives (Jackson Wisconsin, USA). Penicillin-streptomycin, Dulbecco's modified Eagle's medium, and Tygon® S3 (E-3603, ¼″ ID×⅜″ OD× 1/16″) Laboratory Tubing obtained from Thermo Fisher Scientific (Waltham MA, USA). Fetal Bovine Serum and Tygon® E-Lab (E-3603, ⅜″ ID×½″ OD) purchased from VWR (Atlanta, GA USA). Human fibrinogen was purchased from Innovative Research (Novi, MI). Trypsin-EDTA was purchased from Corning (Manassas, VA). Calcium and magnesium-free PBS (1×) was purchased from Corning Incorporated. Drabkin's reagent was purchased from Ricca Chemical Company. High-density polyethylene (HDPE) was purchased from eBay (0.030″×12″×21.5″). Human umbilical vein endothelial cells (ATCC CRL-2522), Staphylococcus aureus (ATCC 6538), and Escherichia coli (ATCC 25922) were procured from American type culture collection (Manassas VA, USA). Collagen Aggregation Reagent 385 and Chrono-Log optical aggregometer was obtained from Chrono-log Corporation (Chrono-PAR #385 Havertown, PA). New Zealand white rabbits were obtained from Charles River Laboratories (2.5-3.5 kg, Wilmington, MA). Enrofloxacin supplied by Norbrook Inc (Enroflox, Lenexa, KS). Meloxicam supplied by Boehringer Ingelheim (Metacam, Duluth, GA). Pentobarbital sodium and phenytoin sodium purchased from Virbac (Euthasol, Fort Worth, TX).

### General Considerations

The surface wettability of the samples was measured via water contact angles (WCA) and water sliding angles (WSA) using an Ossila Contact Angle Goniometer (Model: L2004A1) at three different locations for each sample. Static WCAs were measured by placing 5 μL water droplets on the surface of the samples. WSAs were evaluated using 10 μL water droplets. Spectrum 3 Fourier Transform Infrared (FTIR) spectrometer (PerkinElmer, Greenville SC, USA) was used to monitor the reaction of the bis-APT-PDMS with IPDI via potassium bromide (KBr) pellet method. Briefly, 200 mg of KBr and 10 μL of sample were mixed and cast into a 7 mm pellet press die to form a pellet under 1.5 tons of applied force. Three independent samples were analyzed per sample type. The NO release was measured using Zysense 280i chemiluminescence nitric oxide analyzer (NOA) (Frederick CO, USA). SNAP leaching from the polymeric substrates and bacteria optical density was measured using a UV-vis spectrophotometer (Thermo Fisher Scientific, Waltham MA, USA). Colorimetric assays were analyzed via a Cytation 5 imaging multi-mode plate reader (BioTek, Synergy MX USA). The bacterial biomass accumulation study was conducted using a BioSurfaces DFR-110-4 Drip Flow bioreactor (Bozeman, MT). Blood cell counts were measured using an Element HT5 Veterinary Hematology Analyzer (HESKA Loveland, Colorado). Anesthesia was maintained throughout the animal study using ADS 2000 ventilator (Engler Engineering Corp. Hialeah, FL). THF used throughout the study was dried using 4 Å molecular sieves. For in vitro cytocompatibility, the following cell lines were procured from the American Type Culture Collection: human fibroblasts (BJ, RRID:CVCL_3653, CRL-2522, ATCC, USA) and human umbilical vein endothelial cells (HUVECs, PCS-100-010, ATCC, USA).

### S-nitroso-N-acetylpenicilamine (SNAP) Synthesis

SNAP was synthesized following previous literature protocol [25]. First, 5 g of NAP was dissolved in 60 mL methanol, 5 mL H₂SO₄, and 20 mL HCl while stirring. A sodium nitrate solution (125 mg/mL) was then added dropwise, and N₂ gas was blown over the NAP-containing solution. Once the NAP dissolved, the stirring was stopped, and the mixture was cooled over ice for 8 h as green precipitate formed. The precipitate was collected via vacuum filtration, washed with cold DI water, and stored overnight in a vacuum desiccator to obtain the SNAP crystals. The as-obtained SNAP was protected from light and stored in a −20° C. freezer. The purity was determined via moles of NO released per mole of SNAP using chemiluminescent NO analysis. SNAP with >90% purity was used for subsequent experiments.

### Fabrication of the Different Organogel Derived Slippery Polymeric Films

Fabrication of PVC-SNAP blended polymer films: First, 1 g of PVC was dissolved in 20 mL of THF via stirring on a magnetic hot plate and 4% TOTM was added as the plasticizer. Similarly, the PVC-SNAP films were prepared by blending 20 wt % SNAP with the PVC-THF solution. The PVC-THF solution with and without SNAP was cast into individual Teflon molds (6×4 cm) to allow the THF evaporate overnight in a fume hood. The resultant PVC and PVC-SNAP films were rinsed with DI water and dried in a vacuum desiccator for an additional 24 h to ensure the removal of any residual solvent.

### Fabrication of the Organogel Coated and Lubricant Infused PVC Substrates

To prepare the organogel coating, bis-APT-PDMS (0.28 mmol) and THF (4 mL) were mixed in a 20 mL glass vial followed by stirring for 10 min at ambient conditions. Thereafter, IPDI (0.28 mmol) was added dropwise to the organogel solution followed by stirring at 50° C. for 12 h under inert conditions. Following the reaction, the PVC and PVC-SNAP films were dip coated with the organogel solution twice, with 5 min in between each coat followed by 24 h drying in the fume hood (PVC-OGL and PVC-SNAP-OGL, respectively). The coated substrates were then transferred to a vacuum desiccator for an additional 24 h to remove any residual solvent. Thereafter, the PVC-OGL and PVC-SNAP-OGL films were submerged in silicone oil (100 cSt) for 24 h to obtain PVC-LIS and PVC-SNAP-LIS samples. All sample abbreviations and detailed description are denoted in Table 1.

TABLE 1

| Sample Type | Sample Description |
|---|---|
| PVC | PVC film with 4 wt % plasticizer |
| PVC-OGL | PVC dip-coated with the organogel solution (2x) |
| PVC-LIS | PVC-OGL with 100 cSt silicone oil infused |
| PVC-SNAP | PVC film with 20 wt % SNAP blended |
| PVC-SNAP-OGL | PVC-SNAP dip-coated with the organogel solution |
| | (2x) |
| PVC-SNAP-LIS | PVC-SNAP-OGL with 100 cSt silicone oil infused |

### Analysis of Nitric Oxide Release

The NO release from PVC-SNAP, PVC-SNAP-OGL, and PVC-SNAP-LIS was measured in physiological conditions. First, 3 mL of PBS (10 mM, pH 7.4) with EDTA (100 μM) was added to an amber glass sample chamber. EDTA was added to the solution to chelate trace metal ions to prevent the catalysis of SNAP. The chamber was then placed in a water bath at 37° C. and flushed with N₂ sweep gas as the chamber equilibrated. PVC-SNAP cylindrical sample (6 mm×1 mm) was then placed in the chamber as N₂ was constantly bubbled through at a rate of 200 mL/min to carry the NO released from the sample into the NOA detection chamber. The NO released from the sample reacts with ozone in the reaction chamber and a photon is emitted. The photon is then amplified by a photomultiplier tube and the signal is detected via chemiluminescence detection chamber and converted into the concentration of NO (ppb). The sample surface area was used to normalize the NO release and flux values were obtained (×10⁻¹⁰ mol min⁻¹ cm⁻²). Samples were stored in PBS with EDTA and incubated at 37° C. to measure the NO release at different time points. The same protocol was followed for PVC-SNAP-OGL and PVC-SNAP-LIS samples (n=3).

### Investigation of SNAP Leaching

SNAP leaching from PVC-SNAP, PVC-SNAP-OGL, and PVC-SNAP-LIS was measured in physiological conditions. First, samples were submerged in an amber vial containing 1 mL of PBS (10 mM, pH 7.4, 100 μM EDTA) and incubated at 37° C. Thereafter, the absorption of the leachates for each sample type was measured at 340 nm at various time points (4, 8, 12, 24, and 48 h) to quantify the SNAP leached over time. The sample was submerged in a fresh 1 mL aliquot and incubated between readings. A standard curve of known SNAP concentrations was used to determine the concentration of SNAP in the leachate.

### Durability of PDMS-IPDI Coating

The PVC-LIS and PVC-SNAP-LIS films were exposed to different environmental conditions to investigate the durability of the organogel coating and the sustenance of the slippery behavior. Adhesive tape was repeatedly adhered and peeled off the surface of the composites and the slippery behavior was evaluated over repeated adhesion cycles through the WSA.

Heat Exposure. The slippery behavior of the PVC-LIS substrates was evaluated through the WSA at room temperature, physiological temperature (37° C.), and 100° C. over a 48 h period.

Sterilization. Common sterilization methods consist of UV irradiation for 30 min. The PVC-LIS and PVC-SNAP-LIS substrates were exposed to UV irradiation at 365 nm for 30 min and the slippery property was evaluated through the WSA.

Self-Healing Analysis of Coating. The self-healing behavior of the PVC-LIS and PVC-SNAP-LIS substrates was measured under an optical microscope. First, a blade was used to make an incision in the organogel layer of the substrates, and the incision site was monitored over a 24 h period or until the cut had disappeared entirely at room temperature and 37° C.

Storage stability. The PVC-LIS and PVC-SNAP-LIS substrates were kept at room temperature for 60 days followed by investigation of the embedded slippery property.

### Cytocompatibility Assessment

Cell viability was assessed following exposure to leachates from PVC, PVC-SNAP, PVC-LIS, and PVC-SNAP-LIS samples per ISO 10993-5 standards of assessing indirect in vitro cytotoxicity. Samples were first sterilized under UV in a biosafety cabinet for 15 min. Following the sterilization process, samples were submerged in 1 mL of complete DMEM supplemented with 1% penicillin-streptomycin and 10% FBS, shielded from light, and incubated at 37° C. for 24 h.

Human umbilical vein endothelial cells (HUVEC) were cultured in a humidified incubator to 70% confluency in treated tissue culture flasks with supplemented DMEM at 37° C. and 5% CO₂. Cells were then harvested via trypsinization and seeded in a treated culture-grade 96-well plate at a density of 8,000 cells/well in DMEM overnight to ensure cell attachment. Afterwards, the spent media was replaced with the leachates collected from samples and incubated for an additional 24 h. 100 μL of fresh DMEM was added to cell control wells. An MTT assay was used to determine the relative cell viability. Briefly, the cell media was replaced with 100 μL of 0.5% MTT in sterile calcium and magnesium free (CMF) PBS and incubated for 3 h. The MTT supernatant was then removed from the wells and DMSO was used to dissolve the formazan crystals. The absorbance of each well was read at 540 nm using a plate reader.

Relative cell viability was determined using the absorbance of the untreated wells as the control and assigning it 100% viability. Using the following equation, viability for all sample types was calculated.

$$Relative Cell Viability ( \% ) = \frac{Sample Absorbance}{Control Absorbance} \times 100 \%$$

### Antibacterial Efficacy

The antibacterial activity of PVC-SNAP-LIS was compared to PVC, PVC-SNAP, and PVC-LIS via 48 h biofilm analysis against gram-positive S. aureus and gram-negative E. coli using a BioSurfaces DFR-110-4 Drip Flow bioreactor. Bacteria (E. coli or S. aureus) were inoculated in LB broth and incubated on a bacterial shaker plate at 120 rpm at 37° C. until a log growth phase was reached. The bacterial suspension was then washed with sterile PBS and diluted to ~10⁷ colony forming units (CFU) in sterile PBS.

Samples were UV sterilized in a biosafety cabinet for 15 min on each side. The dilute bacterial suspension was implemented into a 2 g/L LB solution and added into the respective reactor channels that contained the samples. For initial biofilm formation, a 6 h batch phase was conducted in static conditions. Upon completion of the batch phase, commencement of the continuous phase was conducted for 48 h with LB media supplementation at a flow rate of 0.8±0.03 mL min⁻¹ at 37° C. and a bioreactor angle of 10°. Upon completion of the 48 h study, samples were removed from their respective channels and gently rinsed with sterile PBS to remove excess unadhered bacterial cells and biofilm formation was quantified using crystal violet assay.

Samples were exposed to 0.4% CV solution in DI water for 15 min at 25° C. to stain the adhered biofilm. Samples were then gently rinsed with sterile PBS (1×) to remove any unadhered stain from the samples. Samples were then exposed to a 30% acetic acid solution in DI water and the absorbance (595 nm) was quantified on a BioTek Cytation 5 plate reader.

### Fibrinogen Adsorption Analysis

Human fibrinogen was used to evaluate the protein adhesion onto the PVC, PVC-SNAP, PVC-LIS, and PVC-SNAP-LIS surface under physiological conditions (37° C., pH 7.4). First, 100 μL of CMF-PBS (1×) was added to each sample well in a 96-well glass bottom plate. Fluorescein isothiocyanate tagged human Fg (FITC-Fg) was mixed 1:10 with purified unlabeled human fibrinogen to obtain a working solution of 4 mg/mL for sample exposure. 100 μL of the working fibrinogen solution was then added to the wells containing samples and CMF-PBS to obtain a final fibrinogen concentration of 2 mg/mL. The samples were protected from light and incubated at 37° C. for an additional 90 min. Afterwards, 100 μL of the fibrinogen solution was removed from the wells and washed sequentially fresh 1×CMF-PBS to remove any non-adsorbed fibrinogen from the surface of the samples. Samples were removed and fluorescent images were obtained using a fluorescence microscope.

### Hemocompatibility Assays

Porcine Blood Collection and Processing. The University of Georgia's Institutional Animal Care and Use Committee have approved all protocols regarding the collection and processing of porcine whole blood (WB). Sodium citrate in PBS (0.109 M) was used as an anticoagulant for the porcine WB draw. The collected WB samples were analyzed using a hematology analyzer (HESKA element HT5) to obtain hemoglobin levels and other relevant cell counts. WB was then centrifuged (277 RCF, 12 min) to fractionate the blood and collect platelet rich plasma (PRP) for each donor sample. Platelet poor plasma (PPP) was obtained following another centrifugation (3082 RCF, 20 min) and pooled from all collected donors. All processing was conducted within 4 h of the sample draw.

Evaluation of Platelet Adhesion. To evaluate the adhesion of platelets onto the PVC, PVC-SNAP, PVC-LIS, and PVC-SNAP-LIS samples, PRP was first diluted to a working platelet count of 2×10⁸ platelets/mL with PPP and then treated with calcium chloride (2.5 mM) to reverse anticoagulation. Cylindrical samples (6 mm×1 mm) were then added to microcentrifuge tubes with 1 mL of the platelet working solution and incubated at 37° C. for 30 min under gentle rocking. Following the incubation period, the samples were gently washed with 1×CMF-PBS to remove non-adhered platelets from the surface and placed in a 2% (v/v) Triton in PBS lysing solution for 30 min at room temperature for equilibration. Roche Cytotoxicity Detection Kit was used to measure the activity of the lactate dehydrogenase (LDH) released into the lysate solution. A standard curve of known platelet concentrations was used to quantify the concentration of platelets adhered to samples. Final values were reported as platelet count adhered per unit of sample surface area (platelet/cm²) (n=6).

Hemolysis Analysis. The hemolytic index of each sample type (n=6) was evaluated in accordance with 10993-4 ISO standard of direct contact in vitro testing. Porcine WB was diluted to a total hemoglobin level of 10 mg/mL with 1×CMF-PBS. A positive control of 7 mL sterile DI water and 1 mL dilute whole blood (DWB) and a blank of 7 mL CMF-PBS and 1 mL DWB were prepared. Negative controls were prepared using high-density polyethylene (HDPE) samples exposed to DWB at a surface area to volume ratio of 3 cm²/mL. Each of the remaining samples were exposed to DWB at the same surface area to volume ratio and all samples were then incubated at 37° C. for 3 h with inversions at every 30 min. Afterwards, the samples were centrifuged (800 rcf, 15 min) and the supernatant was transferred to a well plate and combined in a 1:1 ratio with Drabkin's reagent to measure the total hemoglobin content. Following a 15 min incubation period at room temperature, the absorbance was measured at 540 nm. The following equations were used to calculate the hemolytic index.

$$\% Hemolysis = \frac{{Abs}_{( Sample )} - {Abs}_{( Blank )}}{{Abs}_{( DWB )} - {Abs}_{( Blank )}} \times 100 \%$$

$$Hemolytic Index = \frac{\% {Hemolysis}_{Sample}}{{Hemolysis}_{HDPE}}$$

### Extracorporeal Circuit Loop Fabrication

Extracorporeal circuit (ECC) loops were constructed using one 8 cm long PVC Tygon tube (⅜″ inner diameter) to create a thrombogenicity chamber connected on either end to 16 cm long PVC Tygon tubes (¼″ inner diameter). These segments were first coated three times with high molecular weight PVC in THF (100 mg mL⁻¹) with or without 20 wt % SNAP to obtain PVC-SNAP or PVC control loop segments, respectively. These segments were then left to dry for 24 h in a fume hood. The PVC-SNAP segments were then further coated three times with the organogel coating with 5 min dry time between each coat. Thereafter, the samples were shielded from light and left in fume hood for 48 h for all solvent to evaporate. All ECC loops segments were adhered together using a PVC in THF (50 mg mL⁻¹) solution and dried under ambient conditions for at least 24 h. The PVC-SNAP-OGL connected loop was then filled with silicone oil for 3 h to obtain the final combinatorial PVC-SNAP-LIS loop used in this study. One 16-gauge polyurethane angiocatheter for arterial access and one 14-gauge polyurethane angiocatheter for venous access (Kendall Monoject Tyco Healthcare Mansfield, MA) was attached at either end of the long tube segments for both the PVC and PVC-SNAP-LIS loops. To prevent systemic vasodilation and obtain consistant NO flux throughout the study, the ECC loops were primed with saline solution for 3 h.

### Rabbit Thrombogenicity Model

The anti-thrombogenic property of the PVC-SNAP-LIS combinatorial material was analyzed in vivo using a well-established rabbit extracorporeal circuit model. The University of Georgia Institutional Animal Care and Use Committee approved the animal handling and surgical procedures in accordance with university and federal regulations. The surgical procedures were conducted in collaboration with trained veterinary researchers and technicians. New Zealand white rabbits were used in this study. Intramuscular injections of 3 mg/kg xylazine, 10 mg/kg ketamine hydrochloride and 0.03 mg/kg buprenorphine were used to initially anesthetize the rabbits (2.7-3.5 kg). Respiratory anesthesia was administered at a rate of 1-4% via an isoflurane inhalation gas anesthesia machine and an ADS 2000 small animal ventilator. Blood pressure was maintained via IV fluids of Lactated Ringers given at a rate of 10 ml kg⁻¹ h⁻¹. The rabbits' right carotid artery was cannulated using an 18-gauge IV angiocatheter to collect blood hourly. Blood was collected (5.4 mL) using 3.2% citrate Vacutainers at a 1:9 ratio. Blood was collected prior to placement of the ECC loop and used as a baseline reading. Temperature, heart rate, and blood pressure were closely monitored throughout the entirety of the surgery.

The rabbit left carotid artery and right external jugular vein were isolated and the ECC was placed into position by cannulating the left carotid artery for ECC inflow and the right external jugular vein for ECC outflow. The arterial and venous sides of ECC were unclamped and blood flow through the circuit was monitored via ultrasonic flow probe and flow meter. Systemic anticoagulants were not administered to maintain normal physiological conditions. Blood function and composition were monitored over the 4 h study via hourly blood draws. After 4 h of blood flow, the circuits were clamped and removed from the rabbit, rinsed with 20 mL of saline solution and drained. All animals received a dose of 400 U/kg sodium heparin to prevent necrotic thrombosis prior to Euthasol administration. Following the study, images were taken down the lumen of the thrombochamber to assess patency and the surface area of thrombus formation was analyzed using imaging software. The thrombus formation was then removed and weighed to obtain clot mass.

### Whole Blood Composition Analysis

Rabbit whole blood was collected hourly throughout the duration of the 4 h in vivo extracorporeal circuit model. The platelet concentrations in the hourly blood draws were measured using an Element HT5 Veterinary Hematology Analyzer.

### Platelet Aggregometry

Platelet aggregation analysis was conducted using previously a described method [26]. Briefly, the whole blood collected hourly from the rabbit was centrifuged (225 RCF, 5 min) to collect platelet rich plasma (PRP). Platelet poor plasma (PPP) was then collected via further centrifugation at 1500 RCF for 10 min. The percentage of platelet aggregation was analyzed using Born's turbidimetric method using a Chrono-Log optical aggregometer. Platelet rich plasma was diluted to 2×10⁸ platelets mL⁻¹ using the collected PPP and 500 μL of the dilute solution was added to glass cuvettes with a magnetic stir bar. Platelet poor plasma was used as a blank. Platelets in the dilute PRP were then activated using 40 μg/mL of collagen and data was recorded using the Chrono-Log Aggrolink software. The degree of platelet aggregation in the PRP sample was determined 7 min after collagen stimulation.

### Sample Preparation for SEM Analysis

PVC-SNAP-LIS Coating Thickness: The thickness of the PVC coated and PVC-SNAP-LIS coated Tygon™ loops were analyzed via SEM imaging of the cross-section of the loop inner lumen. The samples were cut and placed on aluminum mounts to view the cross-section using double-sided black carbon adhesive and 10 nm of Au—Pd powder was sputter coated onto the surface using a Leica EM ACE200 sputter coater (Buffalo Grove, IL USA). Surface imaging of the tubing sections was done with an accelerating voltage of 20 kV and using a Thermo Fisher Scientific Teneo Field Emission SEM. Image J was then used to measure the coating thickness of the PVC-SNAP-LIS loops.

ECC Sample Fixation following Blood Exposure: Following the 4 h ECC study, samples from the thrombogenicity chamber of PVC and PVC-SNAP-LIS were submerged in 2% glutaraldehyde overnight. The samples were then incubated with agitation thrice in 0.1 M HEPES buffer for 5 mins. The samples were then dehydrated via submersion in methanol solutions (50%, 70%, 95%, and 100% methanol) twice at each concentration for 10 min. Finally, samples were dried by a series of bis(trimethylsilyl)amine (HDMS) washing steps (66% ethanol 33% HDMS, 50% ethanol 50% HDMS, 33% ethanol 66% HDMS, and 100% HDMS) for 10 min at each step. After chemical drying, the samples were prepared as mentioned above with the inner lumen facing upwards.

### Statistical Significance

Data obtained is presented as mean±SD with a sample size of 3 unless otherwise stated. Statistical significance was analyzed using GraphPad Prism software and PVC-SNAP-LIS data were compared with controls using an ordinary one-way ANOVA test followed by Turkey's test. Values of p<0.05 were considered statistically significant.

### Results and Discussion

### Synthesis and Characterization of Slippery, Self-Healable Organogel Coating

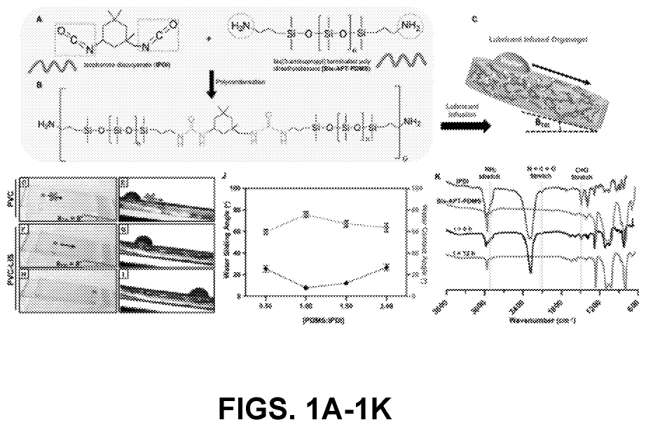

In this work, bis(3-aminopropyl) terminated poly dimethylsiloxane (bis-APT-PDMS) and isophorone diisocyanate (IPDI) was covalently cross-linked through ureido bonds followed by lubricant infusion to obtain a slippery organogel coating on medical grade polymers as shown in FIGS. 1A-C. PVC substrates were chosen for the experiments as it is one of the most widely used polymeric materials for biomedical applications and is not innately compatible with silicone oil. Thus, it is important to impart the lubricant-infused, antifouling properties to PVC [33,34].

As shown in FIGS. 1D-E, the water droplets pinned onto the PVC and organogel coated PVC substrates (abbreviated as PVC-OGL) at a sliding angle of 8° due to the imperfect solid-liquid interface. However, infusion of the silicone oil led to the sliding of the water droplet from the surface of the lubricant infused organogel coating on PVC abbreviated as PVC-LIS with water sliding angle (WSA)<10⁰ (FIGS. 1F-I) due to the presence of the intrinsically smooth and low friction lubricant layer [12, 35, 36]. Static water contact angle (WCA) measurements were used to evaluate the surface wettability of PVC, PVC-OGL, PVC-LIS substrates without any noticeable difference. It was observed that the WCA at the liquid-liquid interface on PVC-SNAP-LIS slightly decreased compared to the solid-liquid interface of the PVC and PVC-OGL composites (74±1.97° compared to 81±1.25° and 76±2.76°, respectively) owing to the presence of the infused lubricant [13].

The static WCA and WSA of the organogel coating on PVC after liquid infusion was investigated with respect to the varying molar ratios of bis-APT-PDMS and IPDI (Table 2).

TABLE 2
The molar concentration of Bis-APT-PDMS and IPDI used
to develop the organogel coating on PVC and the corresponding
water contact angles and water sliding angles.

| Molar ratio | | | | |
|---|---|---|---|---|
| [Bis-APT- | Bis-APT- | | | |
| PDMS:IPDI] | PDMS | IPDI | WCA | WSA |
| 2.0 | 0.28 mmol | 0.14 mmol | 63.67 ± 3.30° | 26.67 ± 2.88° |
| 1.5 | 0.28 mmol | 0.21 mmol | 67.03 ± 3.06° | 12.00 ± 2.00° |
| 1.0 | 0.28 mmol | 0.28 mmol | 75.63 ± 2.26° | 7.67 ± 3.00° |
| 0.5 | 0.28 mmol | 0.56 mmol | 59.53 ± 1.95° | 25.33 ± 2.49° |

As shown in FIG. 1J, equimolar concentration of bis-APT-PDMS and IPDI yielded the optimum WCA and WSA of 76±2.76° and <10°, respectively. Thus, this concentration was used to develop the organogel coating infused with silicone oil on different polymeric substrates and extended for conducting various experiments. The progress of the polycondensation reaction between bis-APT-PDMS and IPDI was monitored through FTIR analysis as shown in FIG. 1K. The NCO stretching at 2260 cm⁻¹ from the isocyanate groups of IPDI was evident in the initial reaction mixture of bis-APT-PDMS and IPDI [37]. Following the reaction (after 12 h), the NCO stretching peak disappeared entirely and a new C═O stretching vibration at 1630 cm⁻¹ was observed, indicating the successful formation of the ureido bond [38]. Thus, the organogel layer was successfully fabricated and characterized without the use of complex material modifications to obtain a slippery coating on polymers for prospective antibiofouling applications.

Integrating Bioactive Nitric Oxide (NO) Release with Slippery Organogel Coating

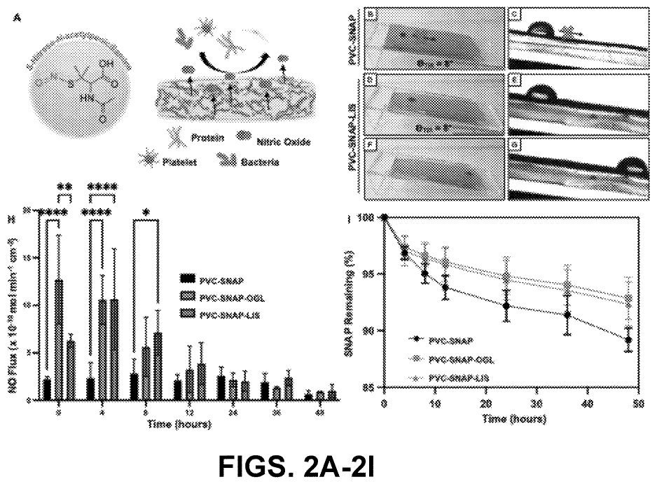

Nitric oxide is an endogenously produced gasotransmitter molecule responsible for preventing thrombosis and bacterial infections in the body [39-41]. In blood vessels, endothelial cells release NO which prevents platelet adhesion and serves as a vasodilator. Immune cells utilize NO release for eradication of pathogens. These antibacterial and anti-thrombotic properties have led to the incorporation of NO donor molecules into biomaterials[42]. Commercial silicone tubing was swelled with NO releasing small molecule followed by silicone oil infusion to obtain a slippery surface with controlled NO release and antifouling properties [13]. This swelling method, however, is limited to substrates having a siloxane-based composition which excludes other medically relevant materials and lacks a self-healing component making the material susceptible to irreparable damage resulting in device failure.
the as-fabricated siloxane-based organogel coating infused with oil as the passive antifouling pathway was integrated with NO-releasing medical grade polymers to achieve overall antibiofouling effects in vitro and in vivo. Primarily, the NO donor molecule S-Nitroso-N-acetylpenicillamine (SNAP) was blended into a PVC polymer following previously reported methods to obtain the SNAP-blended PVC substrates (abbreviated as PVC-SNAP, FIG. 2A)[33]. The PVC-SNAP inherently lacked any slippery characteristics as shown in FIGS. 2B-C. The application of the organogel coating on PVC-SNAP (abbreviated as PVC-SNAP-OGL) followed by lubricant infusion (abbreviated as PVC-SNAP-LIS) continued to exhibit excellent slippery behavior as shown in FIGS. 2D-G with WSA of <10°.

Slippery liquid infused porous surfaces (SLIPS) have garnered attention due to the ability to introduce a molecularly smooth and defect-free liquid interface for foulant repellence [6,27,28]. This passive antibiofouling approach utilizes micro/nano surface features and low surface energy chemical modifications to stably retain the infused lubricant [29-31]. There must be chemical compatibility between the underlying substrate and lubricating liquid to ensure the substrate is stably wetted by the infused lubricant. However, complex fabrication processes to build the surface topography that is also susceptible to mechanical/physical damage have paved the way for organogels infused with organic liquids/lubricants. The surface chemical and physical properties of organogels can be easily manipulated by tuning the infused organic liquid/lubricant [32]. In that context, we exploited the concept of organogels to develop organogel coatings without demanding any topographical needs that can stably hold the infused lubricating layer for potential blood-contacting applications.

The degradation of the S—N bond in SNAP in the presence of heat, light, metal ions, or enzymes generates a thiyl radical and the free radical NO. The localized release of NO from biomaterials exhibits enhanced hemocompatibility and prevents biofilm formation due to its anti-thrombotic and antibacterial activity [43-45]. The NO release from the fabricated PVC-SNAP, PVC-SNAP-OGL, and PVC-SNAP-LIS was assessed over 48 h at physiological conditions using the gold standard chemiluminescent NOA (FIG. 2H). The PVC-SNAP had an initial NO release of 2.21±0.27(×10⁻¹⁰ mol min⁻¹ cm⁻²) and the flux steadily decreased to 0.62±0.37 (×10⁻¹⁰ mol min⁻¹ cm⁻²) after 48 h. The PVC-SNAP-OGL exhibited a significantly higher initial NO flux of 12.7±4.6(×10⁻¹⁰ mol min⁻¹ cm⁻²) as compared to the PVC-SNAP but after 4 h there was no significant difference. The NO release after 48 h for PVC-SNAP-OGL was 0.89±0.05 (×10⁻¹⁰ mol min⁻¹ cm⁻²). The higher initial NO flux in the PVC-SNAP-OGL can be attributed to the siloxane-based organogel coating which is well-known to repel water yet exhibit high water vapor permeability, and such materials have been used in medical applications, moisture regulation, textile fabrication, and membrane distillation [46-48]. The moisture permeable organogel coating is responsible for catalyzing the initial release of NO from the SNAP at the PVC-SNAP-OGL interface, resulting in a higher initial release compared to PVC-SNAP. The PVC-SNAP-LIS exhibited an initial NO flux of 6.2±0.68 (×10⁻¹⁰ mol min⁻¹ cm⁻²), which was higher than PVC-SNAP but significantly lower than PVC-SNAP-OGL. The increased NO release from PVC-SNAP-LIS compared to the PVC-SNAP can be attributed to the incorporation of the moisture permeable organogel layer, however, the infusion of silicone oil acts as a barrier to prevent the rapid hydration of the underlying PVC substrate, which reduces the NO flux compared to PVC-SNAP-OGL [13]. The flux of PVC-SNAP-LIS remained significantly higher than the PVC-SNAP samples for 8 h, which is crucial in preventing the initial biofouling on a device surface and subsequent biofilm formation. Even after 48 h, the NO release remained slightly higher than PVC-SNAP and PVC-SNAP-OGL composites at 0.99±0.68 (×10⁻¹⁰ mol min⁻¹ cm⁻²) which is physiologically known to exhibit antibacterial properties.

The leaching of the SNAP molecule from the polymeric network can induce cellular toxicity at high levels and reduce the longevity of localized NO release [49]. The SNAP remaining inside the underlying bioactive polymer after 48 h was ~90% for PVC and >90% for the PVC-SNAP-OGL and PVC-SNAP-LIS samples. The addition of the hydrophobic organogel coating and silicone oil on the NO-releasing PVC polymer minimized the SNAP leaching from the substrate by serving as a barrier to prevent the diffusion of the SNAP molecule from the bulk polymer as shown in FIG. 2I [25,44]. Thus, the combinatorial polymeric material demonstrated controlled NO release while minimizing SNAP leaching for prolonged and efficient antifouling properties.

### Evaluation of the Self-Healing Performance of the Slippery Organogel Coating

Common approaches to achieve self-healing polymers are mainly based on dynamic covalent interactions which can enhance the thermal and mechanical properties; however, such approaches require the application of external stimuli such as heat or UV irradiation to achieve self-healing [50-52]. For biomedical applications, self-healing polymeric networks that do not require the use of external stimuli are desirable as the use of excessive heat or UV irradiation can cause harm to the surrounding tissue and limits the loading of stimuli-sensitive drug molecules[53].

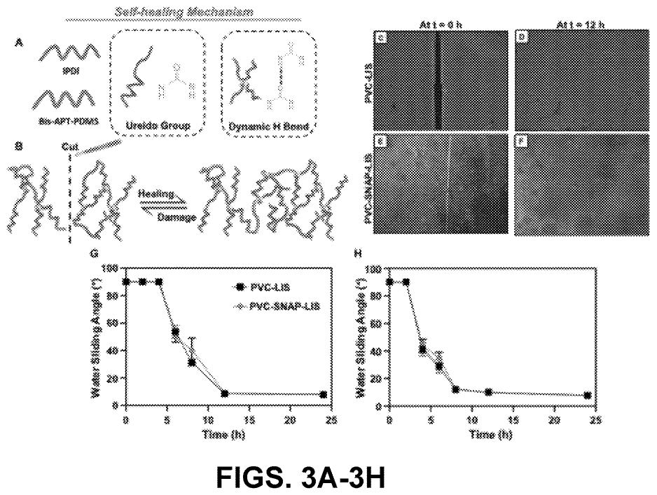

The self-healing performance of the organogel layer was evaluated for the PVC-OGL, PVC-LIS, PVC-SNAP-OGL, and PVC-SNAP-LIS. As represented in FIG. 3A-B, a blade was used to make an incision in the organogel coating, and the incision site was monitored at room temperature over a 24 h period using an optical microscope. After 24 h, the cut had completely disappeared for the PVC-OGL and PVC-SNAP-OGL samples. The incorporation of SNAP in the underlying PVC polymer did not affect the self-healing behavior of the organogel layer. The incision in the PVC-LIS (FIGS. 3C-D) and PVC-SNAP-LIS (FIG. 3E-F) organogel layer completely healed after 12 h as validated through optical microscopy images. The lubricant swelled organogel layer demonstrated faster autonomous healing as the infusion of silicone oil swells the polymer network which can enhance the fluidity of the polymer chains and promote association of the disassociated hydrogen bonds following the incision [56]. The slippery behavior of the PVC-LIS and PVC-SNAP-LIS composites was evaluated during the incision-healing cycle through WSA over 24 h as shown in FIG. 3G. The water droplet was pinned at the incision site directly after the cut, however, after 12 h at room temperature, the water droplet was able to slide off at sliding angles <10°. The slippery property was successfully restored following room temperature self-healing. Further, the effect of physiological temperature on the self-healing behavior of PVC-LIS and PVC-SNAP-LIS was evaluated. Following an incision in the organogel layer, the samples were stored at 37° C. and WSAs were measured over 24 h as shown in FIG. 3H. The PVC-LIS and PVC-SNAP-LIS substrates had a water sliding angle <10° after only 8 h, indicating that an increase in temperature enhanced the rapid self-healing of the lubricant infused organogel layer due to the highly mobile hydrogen bonds and restored the slippery behavior on the surface. Therefore, damage to the surface of the as-fabricated slippery anti-biofouling substrate is capable of self-healing at room temperature and this behavior is enhanced at physiological temperature.

### Durability Analysis of the Embedded Slippery Property

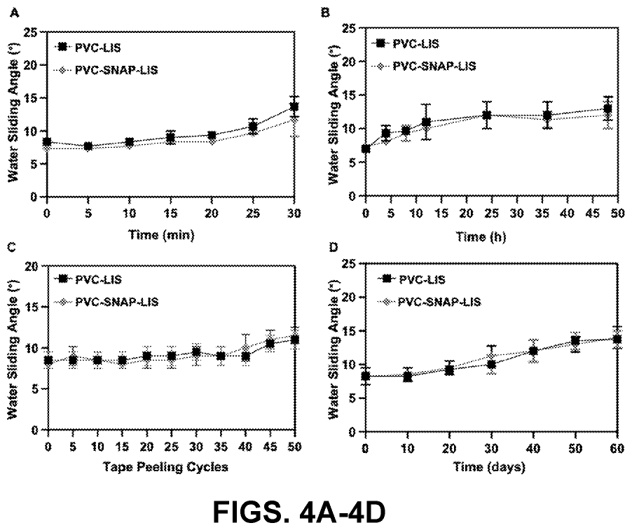

Functional coatings on medical devices are desirable to combat biofouling. Hence, it is crucial that these coatings withstand handling and storage conditions, as well as sterilization methods. The durability of the embedded slippery property in the organogel coating was evaluated in-detail under different abrasive conditions. First, PVC-LIS and PVC-SNAP-LIS were exposed to UV irradiation at 365 nm for 30 min which is a common sterilization method prior to using medical devices for clinical applications. The WSA measured at different time points revealed that both t PVC-LIS and PVC-SNAP-LIS retained the embedded slippery characteristics with WSA<15° exposure as shown in FIG. 4A. The SNAP incorporated in polymers has previously been shown to remain stable even after UV exposure and other sterilization methods, thus, SNAP degradation is ruled out [57]. Next, PVC-LIS and PVC-SNAP-LIS were immersed in phosphate buffer (PBS) for 48 h and FIG. 4B depicts the unperturbed slippery property of the organogel coating with WSA<15°.

To evaluate the efficacy of the organogel coating to withstand handling, a repetitive tape-peeling test was performed on the PVC-LIS and PVC-SNAP-LIS substrates. As shown in FIG. 4C, the WSA remained <10° even after repetitive tape peeling, which can be attributed to the self-healing and self-replenishing nature of the oil-infused organogel coating. Further, PVC-LIS and PVC-SNAP-LIS were stored at room temperature shielded from light to examine the stability of the infused silicone oil within the organogel coating. As depicted in FIG. 4D, the WSA remained <15° up to 60 days demonstrating the retention of the silicone oil within the organogel coating which can be attributed to the chemical compatibility between the lubricant and the organogel. Furthermore, PVC-LIS was subjected to thermal exposure at 37° C. and 100° C. for 48 h and it was observed that the slippery property remained unaffected by such harsh thermal exposure with WSA<15°. Thus, the in-depth durability analysis suggests that the combinatorial material can withstand handling, storage, and sterilization methods demanded for medical applications without compromising the embedded slippery property.

### Anti-Biofouling Efficacy of Slippery Organogel Coated Bioactive Polymer

The endogenous production of NO is known to prevent platelet adhesion in blood vessels, as well as serve as a biocidal agent vital to the immune system. The incorporation of NO-releasing donor molecules in biomaterials has thus been explored for its antifouling efficacy. These NO-releasing materials exhibit excellent platelet and bacteria repellence, however, cannot prevent the adsorption of proteins. This has led to the investigation of integrating the passive, slippery antifouling mechanism with NO-releasing materials to obtain an overall antibiofouling pathway [13]. In this work, the antibiofouling efficacy for PVC, PVC-SNAP, PVC-SNAP-OGL and PVC-SNAP-LIS was examined against bacterial adhesion, protein and platelet repellence in vitro. The lubricant infused organogel coating prevents surface accumulation of biomolecules, while the active release of NO serves as a hemocompatible and biocidal agent. Furthermore, for the first time, this work explored the in vivo efficacy of a slippery organogel coating on a bioactive polymer.

### Evaluation of Biomass Accumulation

Bacterial infections of blood-contacting medical devices accounts for majority of the hospital acquired infections and fatalities in the United States [58]. As antibiotic resistance and failure of antibiotics to penetrate biofilms is a growing concern globally, a broad-spectrum antimicrobial agent having multiple mechanisms of action is desirable that can significantly reduce the development of resistance among microbes [59].

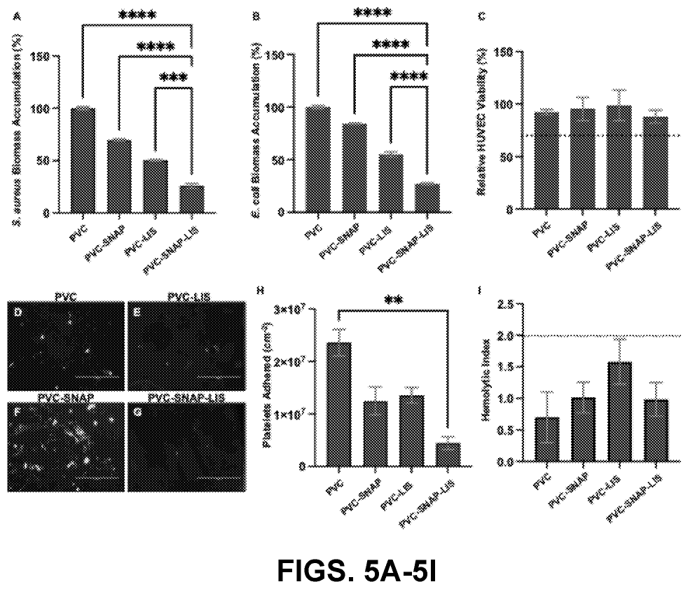

Nitric oxide is produced endogenously, and the small free-radical molecule plays a crucial role in the immune system. It is capable of permeating bacterial cells and biofilms to elicit oxidative and nitrosative stress, resulting in multiple mechanisms of microbial eradication for a broad spectrum of microbes [14]. In this work, the biomass accumulation on PVC, PVC-SNAP, PVC-LIS, and PVC-SNAP-LIS substrates were evaluated following a 48 h drip flow bioreactor study. As shown in FIG. 5A, the PVC-SNAP-LIS combinatorial material exhibited a significant reduction of gram-positive S. aureus biomass accumulation on the surface ~74% in comparison to PVC. A similar trend was observed for PVC-SNAP-LIS against gram-negative E. coli with ~73% biomass reduction compared to PVC as shown in FIG. 5B. The significant reduction in biomass accumulation on PVC-SNAP-LIS can be attributed to the silicone oil infused organogel coating reducing the initial adhesion of the bacteria that ultimately leads to biomass accumulation in combination with the active release of NO and subsequent killing of microbes. The antibacterial efficacy of the combinatorial material demonstrates excellent potential for utilization in healthcare settings.

### Cytocompatibility Assessment

The cytocompatibility of any developed biomaterial needs evaluation for practical utility. Herein, work, the cytocompatibility of PVC, PVC-SNAP, PVC-LIS, and PVC-SNAP-LIS substrates were evaluated in accordance with ISO standards (ISO10993-5: 2009), which demands >70% cell viability for any biomaterial to be classified as cytocompatible. The cytocompatibility of the substrates were evaluated against human umbilical vein endothelial cells (HUVEC) following an indirect contact assessment. Leachate from the samples were collected and exposed to a monolayer of HUVEC cells. Yellow 3-(4,5-dimethylthiazol-2-yl)-2,5-diphenyl-2H-tetrazolium bromide (MTT) assay was then used to quantify the metabolically active cells via formation of purple formazan crystals and assess the cytocompatibility of the samples.

The relative HUVEC viability was determined to be >70% for PVC-SNAP-LIS and all other sample types as shown in FIG. 5C, indicating that the combinatorial polymeric material, as well as the individual components used to fabricate this material, were cytocompatible and safe for further biomedical applications.

### Evaluation of Fibrinogen Adsorption

The adsorption of proteins onto the surface of medical devices is the initial step in surface-induced coagulation that is influenced by surface morphology and chemistry [60]. Minimizing the adsorption of fibrinogen reduces the risk of thrombotic complications which can prolong the use of the medical device and ensure patient safety. Nitric oxide releasing materials have been proven to be ineffective in preventing fibrinogen adsorption as incorporation of NO-releasing donor molecules can increase the surface roughness [61]. Thus, there is a need to introduce a secondary and integrative antifouling mechanism. In that context, lubricant infusion provides a passive adhesion-resistant barrier for reducing the adsorption of proteins and other foulants owing to the presence of the low friction interface and masking any covalent/physical interactions of the beaded proteins with the underlying substrate.

This work combines the slippery organogel coating, which acts as the passive antifouling layer on a NO-releasing to inhibit the adsorption of fibrinogen while capitalizing on the NO release of inhibition of bacteria and platelet adhesion. The PVC, PVC-SNAP, PVC-LIS, and PVC-SNAP-LIS substrates were exposed to fibrinogen solution for a 90 min incubation period followed by fluorescent imaging. As shown in FIG. 5G, PVC-SNAP-LIS exhibited a significant reduction in fibrinogen adhesion as compared to PVC (FIG. 5D) and PVC-SNAP (FIG. 5F) substrates. The reduction in fibrinogen adhesion of PVC-SNAP-LIS is comparable to that of PVC-LIS (FIG. 5E) owing to the presence of the lubricating layer acting as the primary factor responsible for inhibiting protein adhesion. The increased amount of fibrinogen adsorption on the surface of PVC-SNAP substrates can be attributed to the lack of the defect-free lubricant layer, and the incorporation of SNAP in the polymer has been shown to introduce surface roughness, thus promoting fibrinogen adhesion [62].

### Examination of Platelet Adhesion and Hemolysis

Platelet adhesion and subsequent activation on blood-contacting biomaterial surfaces initiates coagulation and eventually leads to thrombus formation [63]. Overcoming this phenomenon is crucial in developing a hemocompatible biomaterial surface. The current work investigates the platelet repellence ability of an organogel coated NO-releasing polymeric substrate. The PVC, PVC-SNAP, PVC-LIS, and PVC-SNAP-LIS samples were exposed to porcine platelet rich plasma (2×10⁸ platelets mL⁻¹) for 90 min at 37° C. under dynamic conditions. The samples were removed and rinsed before quantifying the adhered platelets on the surface. As shown in FIG. 5H, PVC-SNAP-LIS exhibited excellent platelet repellence (>65%) compared to PVC which can be attributed to the ultra-smooth lubricant layer in combination with the active release of nitric oxide from the underlying polymer. The significant reduction in platelet adhesion demonstrates a novel approach for utilizing organogel coatings integrated with active NO release for biomedical applications.

The translation of any material for clinical blood-contacting applications is dependent on the associated red blood cell (RBC) lysis. The hemolytic index of PVC, PVC-SNAP, PVC-LIS, and PVC-SNAP-LIS substrates was assessed following the modified ISO 10993 protocol to determine hemocompatibility of the fabricated material. According to this criterion, a hemolytic index of 0-2% is considered non-hemolytic, 2-5% considered slightly hemolytic, and >5% hemolytic [64]. As shown in FIG. 5I, the hemolytic index of PVC-SNAP-LIS substrate was ~1±0.2%, thus classified as non-hemolytic. All other substrates were also classified as non-hemolytic with a hemolytic index <2%. These results demonstrate the potential to utilize the as-developed polymeric material for future blood-contacting device applications.

### In Vivo Hemocompatibility Analysis

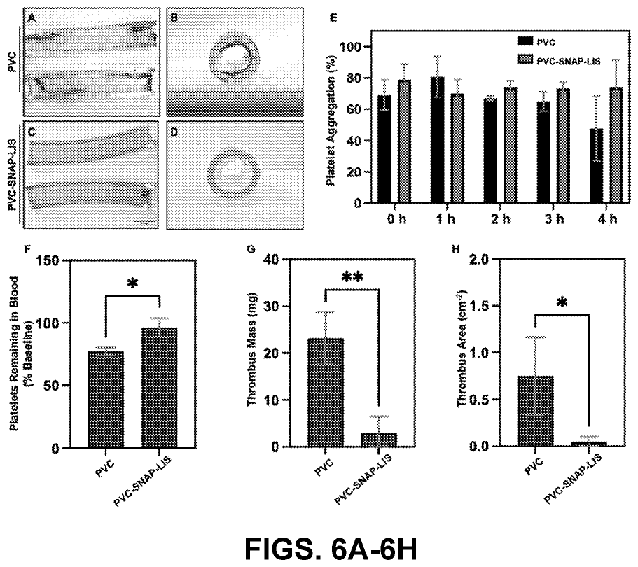

The aforementioned in vitro studies demonstrated the hemocompatibility of the combinatorial slippery coating and these properties were further investigated via a well-established extracorporeal circuit rabbit model [26,41,64,65]. The anti-thrombogenic properties of the PVC-SNAP-LIS coated Tygon loops were tested against the control PVC coated Tygon loops and the thrombus formation within the inner lumen of the thrombogenicity chamber was analyzed after 4 h. Images of the thrombogenicity chambers after the study shown in FIG. 6A-D demonstrate the significant reduction in thrombus formation on the PVC-SNAP-LIS loops compared to the PVC loops. This reduction can be attributed to the passive slippery coating combined with the bioactive NO releasing component.

Platelet response to collagen-induced aggregation in PRP was measured over the duration of the ECC study. The platelet function was maintained for the PVC-SNAP-LIS loop (74±10%) while the platelet aggregation percentage after 4 h was ~30% lower than the baseline measurement for the PVC coated loops (56±12%), as shown in FIG. 6E. The slippery, nitric oxide releasing loop was able to maintain platelet response to ex vivo collagen stimulated aggregation as seen in previous reports [65]. FIG. 6F presents the platelet concentration remaining in the whole blood was also preserved for the PVC-SNAP-LIS loops (~96±7% of baseline remaining) while there was a time dependent reduction in platelets in the blood after the 4 h for the PVC control loops (77±3% of baseline). The reduction of platelet concentration in the whole blood collected from the PVC loop can be attributed to the clot formation within the loop. The thrombus mass formed within the thrombogenicity chamber of the PVC loops was 23.1±2.1 mg which was significantly higher than the thrombus mass formed within the PVC-SNAP-LIS (2.9±3.5 mg) (FIG. 6G). The thrombus mass was reduced by ~87% in the PVC-SNAP-LIS loop compared to the PVC control. The thrombus area was measured via ImageJ software and showed a similar trend in which the clot formed within the PVC thrombogenicity chamber had an area of 0.75±0.41 cm² which was significantly higher than the 0.02±0.001 cm² clot area formed within the PVC-SNAP-LIS material. The reduction in thrombus mass and area for the combinatorial material can be attributed to the NO release from the bioactive component known to attenuate platelet activation and subsequent thrombus formation, as well as the smooth lubricant infused interface [13,65]. The promising results presented from the first-time ECC investigation of a slippery organogel coating on a bioactive polymer demonstrates the efficacy of such coatings for potential use in biomedical applications.

### Substrate-Independent Organogel Coating on Medical-Grade Polymers

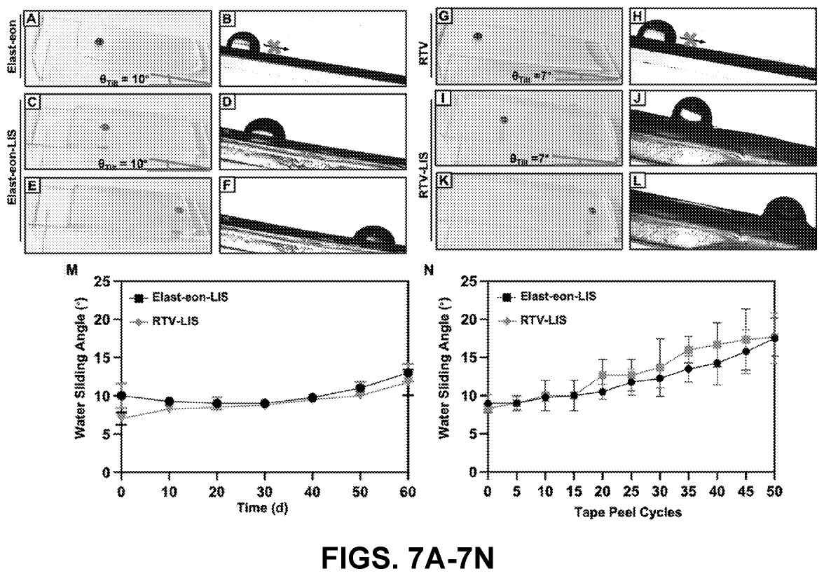

The development of a substrate-independent coating broadens the scope of application, specifically in a medical setting. In this work, the organogel coating was also extended to other polymers, including Elast-eon (silicone polyurethane co-polymer) and RTV (room temperature vulcanized silicone polymer). Polymers with different chemical compositions were used to demonstrate the ability to embed a slippery property on various medically relevant materials to present the significant potential of using such coating to enhance the antifouling efficacy and hemocompatibility of various biomaterials. The commercially available Elast-eon and RTV lacked any inherent slippery characteristics as shown by the pinning of a water droplet on the respective material surface (FIGS. 7A-B, G-H). However, the application of the organogel coating onto Elast-eon and RTV embedded the slippery behavior on the polymer surface with WSA<10⁰ (FIGS. 7C-F, I-L). The water droplet was able to slide off the polymer surface as the addition of the smooth lubricant infused organogel coating was able to reduce the surface tension of the polymer and introduced a liquid-liquid interface capable of repelling the droplet.

These slippery coatings were also evaluated after 60 days in dry room temperature conditions, and the water sliding angles were measured throughout (FIG. 7M). The recorded angles remained <20°, demonstrating that the retention of the lubricant within the organogel layer remains regardless of the underlying polymer. The lubricant infused organogel was also coated on SNAP blended polymers, and the slippery property was analyzed. The water droplet was pinned on the SNAP blended Elast-eon (Elast-eon-SNAP) and RTV (RTV-SNAP). The integration of the lubricant infused organogel allowed for the water droplet to slide at <10°. Additionally, the tape peel test was conducted on the Elast-eon-LIS and RTV-LIS samples to demonstrate the coating durability. It was observed that the slippery behavior remained with a water contact angle <20° throughout the 50 adhesion and peel cycles (FIG. 7N). The ability to stably coat different types of medically relevant polymers broadens the scope and enhances the potential of utilizing the slippery organogel coating for medical applications.

### CONCLUSION

This work reports a slippery organogel coating as a passive antifouling pathway on a bioactive polymer with NO release to combat biofouling in vitro and in vivo. The blending of an antibacterial and anti-thrombotic small NO releasing molecule with different medical-grade polymers such as PVC, Elast-eon, and room temperature vulcanized silicone rubber (RTV) constitutes the bioactive polymer. The polycondensation reaction between bis(3-aminopropyl) terminated poly dimethylsiloxane (bis-APT-PDMS) and isophorone diisocyanate (IPDI) resulted in an organogel coating with oil infusion that was applied onto bioactive polymers with resultant water sliding angles <10°. The organogel coating on the bioactive polymers displayed unperturbed slippery property even after exposure to prolonged UV irradiation (365 nm), room temperature storage up to 60 days, and repetitive adhesive tape test cycles. Further, the slippery organogel coating on the bioactive polymers demonstrated self-healing properties via restoration of the original conformation within 12 hours of physical damage and elevated self-healing under physiological conditions (37° C.). The as-developed cytocompatible and hemocompatible material exhibited excellent platelet (>70%) and protein repellence (>75%), as well as significant reduction in biomass accumulation against gram-negative (E. coli) and gram-positive (S. aureus) bacteria (~73% and ~74%, respectively). Most importantly, this is the first-time report investigating a slippery organogel coating on a bioactive polymer in a 4 h extracorporeal circuit (ECC) rabbit model demonstrating ~87% reduction in thrombus mass. The in vitro and in vivo results pave a novel avenue to extend the current slippery bioactive polymers to combat the biofouling of medical devices.

It should be emphasized that the above-described embodiments of the present disclosure are merely possible examples of implementations and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

[1] I. Banerjee, R. C. Pangule, R. S. Kane, Antifouling Coatings: Recent Developments in the Design of Surfaces That Prevent Fouling by Proteins, Bacteria, and Marine Organisms, Advanced Materials 23(6) (2011) 690-718. https://doi.org/10.1002/adma.201001215.
[2] Q. Song, R. Zhao, T. Liu, L. Gao, C. Su, Y. Ye, S. Y. Chan, X. Liu, K. Wang, P. Li, W. Huang, One-step vapor deposition of fluorinated polycationic coating to fabricate antifouling and anti-infective textile against drug-resistant bacteria and viruses, Chemical Engineering Journal 418 (2021) 129368. https://doi.org/https://doi.org/10.1016/j.cej.2021.129368.
[3] Y. Sun, X. Han, P. Guo, Z. Chai, J. Yue, Y. Su, S. Tan, X. Sun, L. Jiang, L. Heng, Slippery Graphene-Bridging Liquid Metal Layered Heterostructure Nanocomposite for Stable High-Performance Electromagnetic Interference Shielding, ACS Nano 17(13) (2023) 12616-12628. https://doi.org/10.1021/acsnano.3c02975.
[4] M. Ghasemlou, N. Oladzadabbasabadi, E. P. Ivanova, B. Adhikari, C. J. Barrow, Engineered Sustainable Omniphobic Coatings to Control Liquid Spreading on Food-Contact Materials, ACS Applied Materials & Interfaces 16(13) (2024) 15657-15686. https://doi.org/10.1021/acsami.4c01329.
[5] H. Yan, Q. Wu, C. Yu, T. Zhao, M. Liu, Recent Progress of Biomimetic Antifouling Surfaces in Marine, Advanced Materials Interfaces 7(20) (2020) 2000966. https://doi.org/10.1002/admi.202000966.
[6] T.-S. Wong, S. H. Kang, S. K. Y. Tang, E. J. Smythe, B. D. Hatton, A. Grinthal, J. Aizenberg, Bioinspired self-repairing slippery surfaces with pressure-stable omniphobicity, Nature 477(7365) (2011) 443-447. https://doi.org/10.1038/nature10447.
[7] A. Shome, A. Das, A. Borbora, M. Dhar, U. Manna, Role of chemistry in bio-inspired liquid wettability, Chemical Society Reviews 51(13) (2022) 5452-5497. https://doi.org/10.1039/d2cs00255h.
[8] S. Li, F. Zhao, Y. Bai, Z. Ye, Z. Feng, X. Liu, S. Gao, X. Pang, M. Sun, J. Zhang, A. Dong, W. Wang, P. Huang, Slippery liquid-infused microphase separation surface enables highly robust anti-fouling, anti-corrosion, anti-icing and anti-scaling coating on diverse substrates, Chemical Engineering Journal 431 (2022) 133945. https://doi.org/https://doi.org/10.1016/j.cej.2021.133945.
[9] H. H. Tran, Y. Kim, C. Ternon, M. Langlet, D. Riassetto, D. Lee, Lubricant Depletion-Resistant Slippery Liquid-Infused Porous Surfaces via Capillary Rise Lubrication of Nanowire Array, Advanced Materials Interfaces 8(7) (2021) 2002058. https://doi.org/10.1002/admi.202002058.
[10] U. Manna, N. Raman, M. A. Welsh, Y. M. Zayas-Gonzalez, H. E. Blackwell, S. P. Palecek, D. M. Lynn, Slippery Liquid-Infused Porous Surfaces that Prevent Microbial Surface Fouling and Kill Non-Adherent Pathogens in Surrounding Media: A Controlled Release Approach, Advanced Functional Materials 26(21) (2016) 3599-3611. https://doi.org/10.1002/adfm.201505522.
[11] J. Y. Sharahi, T. Azimi, A. Shariati, H. Safari, M. K. Tehrani, A. Hashemi, Advanced strategies for combating bacterial biofilms, Journal of Cellular Physiology 234(9) (2019) 14689-14708. https://doi.org/10.1002/jcp.28225.
[12] A. Das, A. Shome, U. Manna, Porous and reactive polymeric interfaces: an emerging avenue for achieving durable and functional bio-inspired wettability, Journal of Materials Chemistry A 9(2) (2021) 824-856. https://doi.org/10.1039/d0ta10460d.
[13] M. J. Goudie, J. Pant, H. Handa, Liquid-infused nitric oxide-releasing (LINORel) silicone for decreased fouling, thrombosis, and infection of medical devices, Scientific Reports 7(1) (2017). https://doi.org/10.1038/s41598-017-14012-9.
[14] H. K. Nguyen, S. L. Picciotti, M. M. Duke, C. A. Broberg, M. H. Schoenfisch, Nitric Oxide-Induced Morphological Changes to Bacteria, ACS Infectious Diseases 9(11) (2023) 2316-2324. https://doi.org/10.1021/acsinfecdis.3c00415.
[15] Y. Wang, X. Yao, S. Wu, Q. Li, J. Lv, J. Wang, L. Jiang, Bioinspired Solid Organogel Materials with a Regenerable Sacrificial Alkane Surface Layer, Advanced Materials 29(26) (2017) 1700865. https://doi.org/10.1002/adma.201700865.
[16] Z. Wang, B. Yi, M. Wu, D. Lv, M. L. He, M. Liu, X. Yao, Bioinspired Supramolecular Slippery Organogels for Controlling Pathogen Spread by Respiratory Droplets, Advanced Functional Materials 31(34) (2021) 2102888. https://doi.org/10.1002/adfm.202102888.
[17] M. Yan, R. Chen, C. Zhang, Q. Liu, G. Sun, J. Liu, J. Yu, C. Lin, J. Wang, Fully Repairable Slippery Organogel Surfaces with Reconfigurable Paraffin-Based Framework for Universal Antiadhesion, ACS Applied Materials & Interfaces 12(35) (2020) 39807-39816. https://doi.org/10.1021/acsami.0c09915.
[18] S. Wang, M. Qiu, J. Liu, T. Yin, C. Wu, C. Huang, J. Han, S. Cheng, Q. Peng, Y. Li, C. Tie, X. Wu, S. Du, T. Xu, Preshaped 4D Photocurable Ultratough Organogel Microcoils for Personalized Endovascular Embolization, Advanced Materials 35(52) (2023). https://doi.org/10.1002/adma.202308130.
[19] L. Zeng, X. Lin, P. Li, F.-Q. Liu, H. Guo, W.-H. Li, Recent advances of organogels: from fabrications and functions to applications, Progress in Organic Coatings 159 (2021) 106417. https://doi.org/https://doi.org/10.1016/j.porgcoat.2021.106417.
[20] (!!!INVALID CITATION !!! [5-8]).
[21] C. Howell, A. Grinthal, S. Sunny, M. Aizenberg, J. Aizenberg, Designing Liquid-Infused Surfaces for Medical Applications: A Review, Advanced Materials 30(50) (2018) 1802724. https://doi.org/10.1002/adma.201802724.
[22] K. Xing, Z. Li, Z. Wang, S. Qian, J. Feng, C. Gu, J. Tu, Slippery coatings with mechanical robustness and self-replenishing properties as potential application on magnesium alloys, Chemical Engineering Journal 418 (2021) 129079. https://doi.org/https://doi.org/10.1016/j.cej.2021.129079.
[23] C. H. Chien, T. L. Chen, X. Y. Wu, Y. C. Chen, P. Y. Lee, L. Y. Lin, W. L. Wang, H. Y. Hsueh, Bilayer Lubricant-Infused Particulate Films as Slippery Protective Coatings with Durable Anticorrosion and Antifouling Performance, Advanced Materials Interfaces 9(11) (2022) 2102144. https://doi.org/10.1002/admi.202102144.
[24] K. Zhang, X. Shi, J. Chen, T. Xiong, B. Jiang, Y. Huang, Self-healing and stretchable PDMS-based bifunctional sensor enabled by synergistic dynamic interactions, Chemical Engineering Journal 412 (2021) 128734. https://doi.org/https://doi.org/10.1016/j.cej.2021.128734.
[25] E. J. Brisbois, H. Handa, T. C. Major, R. H. Bartlett, M. E. Meyerhoff, Long-term nitric oxide release and elevated temperature stability with S-nitroso-N-acetylpenicillamine (SNAP)-doped Elast-eon E2As polymer, Biomaterials 34(28) (2013) 6957-6966. https://doi.org/https://doi.org/10.1016/j.biomaterials.2013.05.063.
[26] T. C. Major, H. Handa, G. M. Annich, R. H. Bartlett, Development and hemocompatibility testing of nitric oxide releasing polymers using a rabbit model of thrombogenicity, J Biomater Appl 29(4) (2014) 479-501. https://doi.org/10.1177/0885328214538866.
[27] P. Wang, D. Zhang, S. Sun, T. Li, Y. Sun, Fabrication of Slippery Lubricant-Infused Porous Surface with High Underwater Transparency for the Control of Marine Biofouling, ACS Applied Materials & Interfaces 9(1) (2017) 972-982. https://doi.org/10.1021/acsami.6b09117.
[28] S. Zhang, Y. Wang, K. Meng, X. Zheng, Y. Li, H. Chen, Enhanced Anticoagulation of Hierarchy Liquid Infused Surfaces in Blood Flow, ACS Applied Materials & Interfaces 15(48) (2023) 55447-55455. https://doi.org/10.1021/acsami.3c13462.
[29] W. Yan, S. Xue, X. Bin, X. Zhao, W. Zhang, P. Mu, J. Li, Recent advances of slippery liquid-infused porous surfaces with anti-corrosion, Chemical Communications 59(16) (2023) 2182-2198. https://doi.org/10.1039/d2cc06688b.
[30] H. Zhang, Y. Liang, P. Wang, D. Zhang, Design of slippery organogel layer with room-temperature self-healing property for marine anti-fouling application, Progress in Organic Coatings 132 (2019) 132-138. https://doi.org/https://doi.org/10.1016/j.porgcoat.2019.03.020.
[31] X. Zhong, H. Hu, L. Yang, J. Sheng, H. Fu, Robust Hyperbranched Polyester-Based Anti-Smudge Coatings for Self-Cleaning, Anti-Graffiti, and Chemical Shielding, ACS Applied Materials & Interfaces 11(15) (2019) 14305-14312. https://doi.org/10.1021/acsami.8b22447.
[32] M. A. Kuzina, D. D. Kartsev, A. V. Stratonovich, P. A. Levkin, Organogels versus Hydrogels: Advantages, Challenges, and Applications, Advanced Functional Materials 33(27) (2023). https://doi.org/10.1002/adfm.202301421.
[33] R. Pandey, V. Pinon, M. Garren, P. Maffe, A. Mondal, E. J. Brisbois, H. Handa, <i>N</i>-Acetyl Cysteine-Decorated Nitric Oxide-Releasing Interface for Biomedical Applications, ACS Applied Materials & Interfaces 16(19) (2024) 24248-24260. https://doi.org/10.1021/acsami.4c02369.
[34] S. R. Ash, The Importance Of Polyvinyl Chloride In Medical Devices and the Physician's Role in the Choice of Materials, ASAIO Journal 49(3) (2003).
[35] D. C. Leslie, A. Waterhouse, J. B. Berthet, T. M. Valentin, A. L. Watters, A. Jain, P. Kim, B. D. Hatton, A. Nedder, K. Donovan, E. H. Super, C. Howell, C. P. Johnson, T. L. Vu, D. E. Bolgen, S. Rifai, A. R. Hansen, M. Aizenberg, M. Super, J. Aizenberg, D. E. Ingber, A bioinspired omniphobic surface coating on medical devices prevents thrombosis and biofouling, Nature Biotechnology 32(11) (2014) 1134-1140. https://doi.org/10.1038/nbt.3020.
[36] Z. Dong, M. F. Schumann, M. J. Hokkanen, B. Chang, A. Welle, Q. Zhou, R. H. A. Ras, Z. Xu, M. Wegener, P. A. Levkin, Superoleophobic Slippery Lubricant-Infused Surfaces: Combining Two Extremes in the Same Surface, Advanced Materials 30(45) (2018) 1803890. https://doi.org/10.1002/adma.201803890.
[37] F. Da Via, R. Suriano, O. Boumezgane, A. M. Grande, C. Tonelli, S. Turri, Self-healing behavior in blends of PDMS-based polyurethane ionomers, Polymers for Advanced Technologies 33(2) (2022) 556-565. https://doi.org/10.1002/pat.5537.
[38] N. Sun, Z. Wang, Y. Xu, J. Cong, J. Li, L. Bai, P. Huo, Z. Li, Y. Liu, Synthesis of cellulose nanofiber/polysiloxane-polyurea composite materials with self-healing and reprocessing properties, International Journal of Biological Macromolecules 227 (2023) 203-213. https://doi.org/https://doi.org/10.1016/j.ijbiomac.2022.12.168.
[39] J. R. Hall, K. R. Rouillard, D. J. Suchyta, M. D. Brown, M. J. R. Ahonen, M. H. Schoenfisch, Mode of Nitric Oxide Delivery Affects Antibacterial Action, ACS Biomaterials Science & Engineering 6(1) (2020) 433-441. https://doi.org/10.1021/acsbiomaterials.9b01384.
[40] L. Yang, E. S. Feura, M. J. R. Ahonen, M. H. Schoenfisch, Nitric Oxide-Releasing Macromolecular Scaffolds for Antibacterial Applications, Advanced Healthcare Materials 7(13) (2018) 1800155. https://doi.org/10.1002/adhm.201800155.
[41] R. Devine, M. J. Goudie, P. Singha, C. Schmiedt, M. Douglass, E. J. Brisbois, H. Handa, Mimicking the Endothelium: Dual Action Heparinized Nitric Oxide Releasing Surface, ACS Applied Materials & Interfaces 12(18) (2020) 20158-20171. https://doi.org/10.1021/acsami.9b22277.
[42] J. Pant, J. Gao, M. J. Goudie, S. P. Hopkins, J. Locklin, H. Handa, A multi-defense strategy: Enhancing bactericidal activity of a medical grade polymer with a nitric oxide donor and surface-immobilized quaternary ammonium compound, Acta Biomater 58 (2017) 421-431. https://doi.org/10.1016/j.actbio.2017.05.061.
[43] Y. Wo, E. J. Brisbois, R. H. Bartlett, M. E. Meyerhoff, Recent advances in thromboresistant and antimicrobial polymers for biomedical applications: just say yes to nitric oxide (NO), Biomaterials Science 4(8) (2016) 1161-1183. https://doi.org/10.1039/c6bm00271d.
[44] C. G. Feit, M. K. Chug, E. J. Brisbois, Development of S-Nitroso-N-Acetylpenicillamine Impregnated Medical Grade Polyvinyl Chloride for Antimicrobial Medical Device Interfaces, ACS Applied Bio Materials 2(10) (2019) 4335-4345. https://doi.org/10.1021/acsabm.9b00593.
[45] M. C. Jen, M. C. Serrano, R. Van Lith, G. A. Ameer, Polymer-Based Nitric Oxide Therapies: Recent Insights for Biomedical Applications, Advanced Functional Materials 22(2) (2012) 239-260. https://doi.org/10.1002/adfm.201101707.
[46] P. Bian, Y. Wang, T. J. McCarthy, Rediscovering Silicones: The Anomalous Water Permeability of “Hydrophobic” PDMS Suggests Nanostructure and Applications in Water Purification and Anti-Icing, Macromolecular Rapid Communications 42(5) (2021) 2000682. https://doi.org/10.1002/marc.202000682.
[47] J. Sheng, M. Zhang, Y. Xu, J. Yu, B. Ding, Tailoring Water-Resistant and Breathable Performance of Polyacrylonitrile Nanofibrous Membranes Modified by Polydimethylsiloxane, ACS Applied Materials & Interfaces 8(40) (2016) 27218-27226. https://doi.org/10.1021/acsami.6b09392.
[48] G. T. Roman, C. T. Culbertson, Surface Engineering of Poly(dimethylsiloxane) Microfluidic Devices Using Transition Metal Sol-Gel Chemistry, Langmuir 22(9) (2006) 4445-4451. https://doi.org/10.1021/la053085w.
[49] S. I. M. Shishido, A. B. Seabra, W. Loh, M. Ganzarolli de Oliveira, Thermal and photochemical nitric oxide release from S-nitrosothiols incorporated in Pluronic F127 gel: potential uses for local and controlled nitric oxide release, Biomaterials 24(20) (2003) 3543-3553. https://doi.org/https://doi.org/10.1016/S0142-9612(03)00153-4.
[50] Y. Guan, H. Li, S. Zhang, W. Niu, Mechanochromic Photonic Vitrimer Thermal Management Device Based on Dynamic Covalent Bond, Advanced Functional Materials 33(16) (2023) 2215055. https://doi.org/10.1002/adfm.202215055.
[51] J. Dahlke, S. Zechel, M. D. Hager, U. S. Schubert, How to Design a Self-Healing Polymer: General Concepts of Dynamic Covalent Bonds and Their Application for Intrinsic Healable Materials, Advanced Materials Interfaces 5(17) (2018) 1800051. https://doi.org/10.1002/admi.201800051.
[52] M. Burnworth, L. Tang, J. R. Kumpfer, A. J. Duncan, F. L. Beyer, G. L. Fiore, S. J. Rowan, C. Weder, Optically healable supramolecular polymers, Nature 472(7343) (2011) 334-337. https://doi.org/10.1038/nature09963.
[53] C. Jiang, L. Zhang, Q. Yang, S. Huang, H. Shi, Q. Long, B. Qian, Z. Liu, Q. Guan, M. Liu, R. Yang, Q. Zhao, Z. You, X. Ye, Self-healing polyurethane-elastomer with mechanical tunability for multiple biomedical applications in vivo, Nature Communications 12(1) (2021). https://doi.org/10.1038/s41467-021-24680-x.
[54] L. Chen, J. Xu, M. Zhu, Z. Zeng, Y. Song, Y. Zhang, X. Zhang, Y. Deng, R. Xiong, C. Huang, Self-healing polymers through hydrogen-bond cross-linking: synthesis and electronic applications, Materials Horizons 10(10) (2023) 4000-4032. https://doi.org/10.1039/d3mh00236e.
[55] Z. Yang, H. Li, C. Li, X. Lai, X. Zeng, Conductive and room-temperature self-healable polydimethylsiloxane-based elastomer film with ridge-like microstructure for piezoresistive pressure sensor, Chemical Engineering Journal 430 (2022) 133103. https://doi.org/https://doi.org/10.1016/j.cej.2021.133103.
[56] M. Yu, M. Liu, S. Fu, Slippery Antifouling Polysiloxane-Polyurea Surfaces with Matrix Self-Healing and Lubricant Self-Replenishing, ACS Applied Materials & Interfaces 13(27) (2021) 32149-32160. https://doi.org/10.1021/acsami.1c07132.
[57] M. K. Chug, E. J. Brisbois, Recent Developments in Multifunctional Antimicrobial Surfaces and Applications toward Advanced Nitric Oxide-Based Biomaterials, ACS Materials Au 2(5) (2022) 525-551. https://doi.org/10.1021/acsmaterialsau.2c00040.
[58] N. C. T. Dadi, B. Radochove, J. Vargove, H. Bujdekove, Impact of Healthcare-Associated Infections Connected to Medical Devices—An Update, Microorganisms 9(11) (2021). https://doi.org/10.3390/microorganisms9112332.
[59] D. G. J. Larsson, C.-F. Flach, Antibiotic resistance in the environment, Nature Reviews Microbiology 20(5) (2022) 257-269. https://doi.org/10.1038/s41579-021-00649-x.
[60] A. N. O. Nzulumike, E. Thormann, Fibrin Adsorption on Cardiovascular Biomaterials and Medical Devices, ACS Applied Bio Materials 6(7) (2023) 2667-2676. https://doi.org/10.1021/acsabm.2c01057.
[61] S. M. Lantvit, B. J. Barrett, M. M. Reynolds, Nitric oxide releasing material adsorbs more fibrinogen, Journal of Biomedical Materials Research Part A 101(11) (2013) 3201-3210. https://doi.org/10.1002/jbm.a.34627.
[62] M. J. Goudie, E. J. Brisbois, J. Pant, A. Thompson, J. A. Potkay, H. Handa, Characterization of an S-nitroso-N-acetylpenicillamine-based nitric oxide releasing polymer from a translational perspective, International Journal of Polymeric Materials and Polymeric Biomaterials 65(15) (2016) 769-778. https://doi.org/10.1080/00914037.2016.1163570.
[63] Y. Sang, M. Roest, B. De Laat, P. G. De Groot, D. Huskens, Interplay between platelets and coagulation, Blood Reviews 46 (2021) 100733. https://doi.org/10.1016/j.blre.2020.100733.
[64] E. J. Brisbois, T. C. Major, M. J. Goudie, M. E. Meyerhoff, R. H. Bartlett, H. Handa, Attenuation of thrombosis and bacterial infection using dual function nitric oxide releasing central venous catheters in a 9 day rabbit model, Acta Biomaterialia 44 (2016) 304-312. https://doi.org/https://doi.org/10.1016/j.actbio.2016.08.009.
[65] T. C. Major, D. O. Brant, M. M. Reynolds, R. H. Bartlett, M. E. Meyerhoff, H. Handa, G. M. Annich, The attenuation of platelet and monocyte activation in a rabbit model of extracorporeal circulation by a nitric oxide releasing polymer, Biomaterials 31(10) (2010) 2736-2745. https://doi.org/https://doi.org/10.1016/j.biomaterials.2009.12.028.

1. A slippery organogel comprising (a) compound comprising the reaction product between a polysiloxane comprising two or more isocyanate reactive groups and a diisocyanate and (b) a lubricant.

2. The slippery organogel of claim 1, wherein the polysiloxane comprises a linear or branched polysiloxane, wherein each end of the linear or branched polysiloxane has an isocyanate reactive group covalently bonded to each end.

3. The slippery organogel of claim 1, wherein the polysiloxane comprises a polydimethylsiloxane, a polydiethylsiloxane, a polydipropylsiloxane, or a polydiphenylsiloxane.

4. The slippery organogel of claim 1, wherein the polysiloxane has the structure I 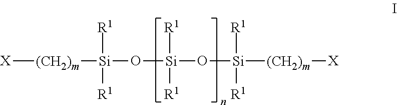
wherein each R¹ is independently selected from alkyl, aryl, alkenyl, hydrogen, alkoxy;
m is an integer from 1 to 10;
n is from about 2,500 to about 27,000; and
X is an isocyanate reactive group.

5. The slippery organogel of claim 4, wherein each R¹ is a methyl group, m is 3, and each X is NH₂.

6. The slippery organogel of claim 1, wherein the diisocyanate has the formula O═C═N—R¹—N═C═O, wherein R¹ is an alkylene group, a cycloalkyl group, an alkylene cycloalkyl group, or an aryl group.

7. The slippery organogel of claim 1, wherein the diisocyanate comprises toluene-2,4-diisocyanate (TDI), diphenylmethane diisocyanate, methylene diphenyl diisocyanate (MDI), hexamethylene diisocyanate (HMDI), 2,2,4-trimethylhexane-1,6-diisocyanate (TMDI), isophorone diisocyanate (IPDI), or 4,4′-diisocyanatodicyclohexylmethane (H12MDI).

8. The slippery organogel of claim 1, wherein a molar ratio of polysiloxane comprising two or more isocyanate reactive groups to the diisocyanate is from 3:1 to 0.5:1.

9. The slippery organogel of claim 1, wherein the lubricant comprises a silicone oil.

10. The slippery organogel of claim 1, wherein the organogel further comprises a nitric oxide releasing compound.

11. The slippery organogel of claim 10, wherein the nitric oxide releasing compound is a S-nitrosothiol compound.

12. The slippery organogel of claim 10, wherein the nitric oxide releasing compound is S-nitroso-N-acetyl-penicillamine, S-nitroso-N-acetylcysteine, S-nitroso-N-acetyl cysteamine, S-nitrosoglutathione, S-nitrosocysteamine-glutathione, methyl S-nitrosothioglycolate, nitrosated cysteine, S-nitroso-N-acetyl-1-cysteine ethyl ester (SNACET), S-nitroso-L-homocysteine, S-nitroso-L-cysteine, S-nitroso-albumin, S-nitrosocaptopril, or any combination thereof.

13. The slippery organogel of claim 1, wherein the slippery organogel further comprises a bioactive agent.

14. An article comprising at least one surface coated with the slippery organogel of claim 1.

15. The article of claim 14, wherein the article comprises a medical device.

16. The article of claim 14, wherein the article is a catheter, a cannula, tubing, a suture, an artificial heart valve, a graft, a stent, circuitry, a joint prostheses, or a dressing.

17. A method for reducing or preventing the growth of microbes on an article, the method comprising applying the slippery organogel of claim 1 to at least one surface of the article.

18. A method for reducing or preventing the formation of a biofilm on an article, the method comprising applying the slippery organogel of claim 1 to at least one surface of the article.

19. A method for reducing or preventing the adhesion of platelets on an article, the method comprising applying the slippery organogel of claim 1 to at least one surface of the article.

20. A method for reducing or preventing the adhesion of a protein on an article, the method comprising applying the slippery organogel of claim 1 to at least one surface of the article.
